(19) United States
(12) Patent Application Publication
(10) Pub. No.: US 2018/0200248 A1
(43) Pub. Date: Jul. 19, 2018

(54) TREATMENT OF HEARING AND BALANCE IMPAIRMENTS WITH REDOX-ACTIVE THERAPEUTICS

(71) Applicant: BIOELECTRON TECHNOLOGY CORPORATION, Mountain View, CA (US)

(72) Inventor: Guy M. Miller, Monte Sereno, CA (US)

(21) Appl. No.: 15/849,550

(22) Filed: Dec. 20, 2017

Related U.S. Application Data
(63) Continuation of application No. 14/174,753, filed on Feb. 6, 2014, now abandoned.
Application No. 14/174,753 is a continuation of application No. 12/919,004, filed on Oct. 21, 2010, now abandoned.
(60) Provisional application No. 61/068,330, filed on Mar. 5, 2008.
(60) Provisional application No. 61/191,198, filed on Sep. 5, 2008.

(51) Int. Cl. A61K 31/495; A61K 31/16; A61K 33/24; A61K 45/06; A61K 31/122

(52) U.S. Cl. CPC A61K 31/495; A61K 31/16; A61K 2300/00; A61K 45/06; A61K 31/122; A61K 33/24

(57) ABSTRACT

Compositions and methods are provided for prophylactic or therapeutic treatment of a mammal for hearing or balance impairments involving neuronal damage, loss, or degeneration, by administration of a therapeutically effective amount of a redox-active therapeutic. Also provided are improved compositions and methods for treatments requiring administration of a pharmaceutical having an ototoxic side-effect in combination with a therapeutically effective amount of a redox-active therapeutic to treat the ototoxicity.

### CROSS-REFERENCE TO RELATED APPLICATIONS

This application claims priority benefit of U.S. Provisional Patent Application No. 61/068,330, filed Mar. 5, 2008, and of U.S. Provisional Patent Application No. 61/191,198, filed Sep. 5, 2008. Those applications are hereby incorporated by reference herein in their entireties.

### TECHNICAL FIELD

The present invention relates to compositions and methods for prophylactic and therapeutic treatment of hearing impairments, particularly for the treatment of noise induced, age-induced and ototoxin-induced hearing impairments involving inner hair cell damage or loss, neuronal damage, loss or degeneration of neurons in a patient, or for the prevention of toxic side effects of ototoxic medications, by administration of redox-active therapeutics. The present invention also relates to compositions and methods for prophylactic and therapeutic treatment of balance impairments.

### BACKGROUND

Hearing impairments are serious handicaps which affect millions of people. Hearing impairments can be attributed to a wide variety of causes, including infections, mechanical injury, loud sounds, aging, and chemical-induced ototoxicity that damages hair cells of the peripheral auditory system and/or the primary afferent neurons in the spiral ganglia that transduce auditory signals from the hair cells to the brain.

The peripheral auditory system consists of auditory receptors, hair cells in the organ of Corti, and primary auditory neurons, the spiral ganglion neurons in the cochlea. Spiral ganglion neurons (“SGN”) are primary afferent auditory neurons that deliver signals from the peripheral auditory receptors, the hair cells in the organ of Corti, to the brain through the cochlear nerve. The eighth nerve connects the primary auditory neurons in the spiral ganglia to the brain stem. The eighth nerve also connects vestibular ganglion neurons (“VGN”), which are primary afferent sensory neurons responsible for balance and which deliver signals from the utricle, saccule and ampullae of the inner ear to the brain.

Noise induced hearing loss (NIHL) can arise under either acute or chronic circumstances. Noise induced hearing loss can give rise to multifarious problems. In addition to the inability to hear certain sounds, especially in the upper registers, individuals experiencing such hearing loss may also experience tinnitus or ringing of the ears. Additionally noise can mechanically irritate the inner ear, giving rise to an inflammatory response characterized by fluid buildup and dampening of sound transmission within the ear. Moreover, excessive noise can also give rise to a neuronal type of hearing loss. In the earlier stages of neuronal hearing loss, the patient experiences a degradation of his ability to discriminate between certain words or to understand certain persons with voices in the upper or lower registers. It has been reported that certain antioxidants, particularly idebenone and Vitamin E therapy offer a potential approach to attenuate noise induced hearing loss (Fetoni, A. R., Neuroreport (2008) Vol 19, No. 3, 277-281). Similarly Trolox has been reported to attenuate noise-induced hearing loss (Yamashita D., Neuroscience (2005), 134:633-643).

Another type of hearing loss is drug-induced or chemically-induced hearing loss (CIHL). Both the vestibular and auditory systems are sensitive to ototoxic drugs, which are detrimental to hearing or balance or both. Ototoxic drugs include therapeutic drugs, antineoplastic agents, contaminants in foods or medicaments, and environmental and industrial pollutants. Ototoxic drugs include the widely used chemotherapeutic agent cisplatin and its analogs (Fleischman et al., Toxicol Appl. Pharmacol. (1975) 33:320-332; Stadnicki et al. Cancer Chemother. Rep. (1975) 59:467-480; Nakai et al., Acta Otarolyngol. (1982) 93:227-232; Berggren et al., Acta Otarolyngol. (1990) 109;57-65; Dublin, Fundamentals of sensorineural auditory pathology. Springfield, Ill.: C. C. Thomas (1976); Hood and Berlin, Contemporary applications of neurobiology in human hearing assessment (Raven Press, N.Y., 1986)), commonly used aminoglycoside antibiotics, e.g. gentamicin, for the treatment of infections caused by Gram-negative bacteria, (Sera et al., Scanning Microsc. (1987) 1 1191:1197; Hinojosa and Lerner, J. Infect. Dis. (1987) 156: 449-455; Bareggi et al., Pharmacol. Res. (1990) 2:635-644), quinine and its analogs, salicylate and its analogs, and loop-diuretics.

Aminoglycoside antibiotics are vital for the treatment of serious bacterial infections. However, in some patients, the antibiotics have severe toxic effects, particularly on the auditory system. The toxic effects of these drugs on auditory cells and spiral ganglion neurons are often the limiting factor for their therapeutic usefulness. For example, antibacterial aminoglycosides such as gentamicins, streptomycins, kanamycins, tobramycins, and the like are known to have serious toxicity, particularly ototoxicity and nephrotoxicity, which reduce the usefulness of such antimicrobial agents (see Goodman and Gilman's The Pharmacological Basis of Therapeutics, 6th ed., A. Goodman Gilman et al., eds; Macmillan Publishing Co., Inc., New York, pp. 1169-71 (1980) or most recent edition). Aminoglycoside antibiotics are generally utilized as broad spectrum antimicrobials effective against, for example, gram-positive, gram-negative and acid-fast bacteria. Susceptible microorganisms include Escherichia spp., Hemophilus spp., Listeria spp., Pseudomonas spp., Nocardia spp., Yersinia spp., Klebsiella spp., Enterobacter spp., Salmonella spp., Staphylococcus spp., Streptococcus spp., Mycobacteria spp., Shigella spp., and Serratia spp.

As implied by the generic name for the family, all the aminoglycoside antibiotics contain aminosugars in glycosidic linkage. Ototoxicity is a dose-limiting side-effect of antibiotic administration. For example, nearly 75% of patients given 2 grams of streptomycin daily for 60 to 120 days displayed some vestibular impairment, whereas at 1 gram per day, the incidence decreased to 25% (U.S. Pat. No. 5,059,591). Auditory impairment was observed: from 4 to 15% of patients receiving 1 gram per day for greater than 1 week develop measurable hearing loss, which slowly becomes worse and can lead to complete permanent deafness if treatment continues. Ototoxicity is also a serious dose-limiting side-effect for cisplatin, a platinum coordination complex that has proven effective on a variety of human cancers including testicular, ovarian, bladder, and head and neck cancer. Cisplatin damages auditory and vestibular systems (Toxicol. Appl. Pharmacol. (1975) 33:320-332; Stadnicki et al. Cancer Chemother. Rep. (1975) 59:467-480; Nakai et al., Acta Otarolyngol. (1982) 93:227-232; Carenza et al. Gynecol. Oncol., (1986) 25:244-249; Sera et al., Scanning Microsc. (1987) 1 1191:1197; Bareggi et al., Pharmacol. Res. (1990) 2:635-644)). Salicylates, such as aspirin, are the most commonly used therapeutic drugs for their anti-inflammatory, analgesic, anti-pyretic and anti-thrombotic effects. Unfortunately, they have ototoxic side effects. They often lead to tinnitus (“ringing in the ears”) and temporary hearing loss (Myers and Bernstein, Arch Otorlarygol. Head Neck Surg. (1965) 82: 483-493. However, if the drug is used at high doses for a prolonged time, the hearing impairment can become persistent and irreversible, as reported clinically (Jarvis, J. Laryngo.l (1966) 80:318-320. Oxitoxicity can also be induced by excitatory neurotoxins such as glutamate and aspartate.

Accordingly, there exists a need for means to prevent, reduce or treat the incidence and/or severity of hearing impairments involving auditory nerves, particularly that arising as an unwanted side-effect of ototoxic therapeutic drugs including cisplatin and its analogs, aminoglycoside antibiotics including gentamicin and analogs, salicylate and its analogs, and loop diuretics. In addition, there exits a need for methods which will allow higher and thus more effective dosing with these ototoxicity-inducing pharmaceutical drugs, while concomitantly preventing or reducing ototoxic effects caused by these drugs. What is needed is a method that provides a safe, effective, and prolonged means for prophylactic or curative treatment of hearing impairments related to nerve damage, loss, or degeneration, particularly ototoxin-induced. In addition there is needed a rapid, reliable, and facile system for testing the effects and mechanisms of ototoxic agents on hearing in animals, including humans, and for testing the efficacy of therapeutics to prevent, reduce or treat these impairments. The present invention provides a method and system to achieve these goals and others as well.

### DISCLOSURE OF THE INVENTION

The present invention is based on the discovery disclosed herein that administration of certain redox-active therapeutics can prevent, or reduce hearing impairments and balance impairments. The impairments are due to inner ear hair cell damage or loss, or neuronal damage, wherein the damage or loss is caused by infection, mechanical injury, aging, noise, acoustic trauma, or chemical-induced ototoxicity. The compounds of the present invention may be administered to promote the protection, survival or regeneration of hair cells and spiral ganglion neurons, thus reversing, enhancing, reducing or preventing hearing loss. Damage to the peripheral auditory system is responsible for a majority of balance deficits (Dublin, Fundamentals of Sensorineural Auditory Pathology (Chapter 3), Springfield, Ill.: Charles C. Thomas 18-103 (1976); Lim, D. J., Am. J. Otolaryngol. 7(2):73-99 (1986) with destruction of vestibular ganglia neurons as a major cause of balance impairments. The present invention also addresses the treatment of balance impairments caused by infections, mechanical injury, loud sounds, aging, and chemical-induced ototoxicity that damage neurons and/or hair cells of the peripheral vestibular systems of the inner ear.

In one embodiment, the invention relates to a method for treating a patient having or prone to having a hearing or balance impairment with a prophylactically or therapeutically effective amount of a redox-active therapeutic, to prevent, reduce or treat the incidence of, or severity of the hearing impairment.

In another embodiment, the invention relates to a method of reversing hearing loss, or recovering or enhancing hearing function with a prophylactically or therapeutically effective amount of a redox-active therapeutic.

In some embodiments the redox-active therapeutic comprises a compound of Formula I, or Formula II, or Formula III, or Formula IV, or Formula V, or Formula VI as described herein. In other embodiments the redox active therapeutic comprises a compound of Formula I, or Formula II, or Formula III, or Formula IV. In other embodiments, the redox-active therapeutic comprises a compound selected from alpha tocopherol quinone, beta tocopherol quinone, gamma tocopherol quinone, alpha tocotrienol quinone, beta tocotrienol quinone, and gamma tocotrienol quinone and mixtures thereof.

In other embodiments the redox active therapeutic comprises a compound of Formula V, as described herein. In other embodiments the redox active therapeutic comprises a compound selected from 2-(3-hydroxy-4-(4-hydroxypiperidin-1-yl)-3-methyl-4-oxobutyl)-3,5,6-trimethylcyclohexa-2,5-diene-1,4-dione, 2-hydroxy-2-methyl-4-(2,4,5-trimethyl-3,6-dioxocyclohexa-1,4-dienyl)butanamide, 2-(4-(4-acetylpiperazin-1-yl)-3-hydroxy-3-methyl-4-oxobutyl)-3,5,6-trimethylcyclohexa-2,5-diene-1,4-dione, N-(2-(dimethylamino)ethyl)-2-hydroxy-2-methyl-4-(2,4,5-trimethyl-3,6-dioxocyclohexa-1,4-dienyl)butanamide, and 2-(3-hydroxy-3-methyl-4-(4-methylpiperazin-1-yl)-4-oxobutyl)-3,5,6-trimethylcyclohexa-2,5-diene-1,4-dione.

In other embodiments, the redox-active therapeutic comprises a compound of Formula VI. In other embodiments the redox-active therapeutic comprises a compound selected from 2-(3-hydroxy-3-methylbutyl)-5,6-dimethyl-3-(4-(trifluoromethyl)phenyl)-cyclohexa-2,5-diene-1,4-dione, 2-(3-hydroxy-3-methylbutyl)-5,6-dimethyl-3-(4-(trifluoromethyl)phenyl)cyclohexa-2,5-diene-1,4-dione, 2-(3-hydroxy-3-methylbutyl)-3,5-dimethyl-6-(4-(trifluoromethyl)phenyl)cyclohexa-2,5-diene-1,4-dione, and 2-(4-chlorophenyl)-6-(3-hydroxy-3-methylbutyl)-3,5-dimethylcyclohexa-2,5-diene-1,4-dione.

In other embodiments, the redox-active therapeutic consists essentially of alpha tocotrienol, beta tocotrienol, gamma tocotrienol or mixtures thereof. In some other embodiments, the redox-active therapeutic is selected from essentially pure alpha tocotrienol, essentially pure beta tocotrienol, essentially pure gamma tocotrienol, essentially pure alpha tocotrienol quinone, essentially pure beta tocotrienol quinone, essentially pure gamma tocotrienol quinone. In some embodiments, the redox-active therapeutic is a natural mixture of tocopherols and tocotrienols extracted from palm oil or cereal grains (such as oat, barley, and rye, rice bran). In some embodiments, the redox-active therapeutic is a mixture of tocopherols and tocotrienol sold by Carotech as Tocomin® or Tocomin® SupraBio™ a series of products containing natural occurring mixture of tocotrienols and tocopherol extracted and concentrated from virgin crude palm oil/palm fruits (Elaeis guineensis).

In other embodiments, the redox-active therapeutic consists essentially Tdebenone, CoQ10, vitamin E, Trolox (6-Hydroxy-2,5,7,8-tetramethylchroman-2-carbonic acid), and mixtures thereof, and the impairment is an ototoxin-induced or inducible impairment.

In one embodiment, the invention relates to a method for treating a patient having or prone to having a noise-induced hearing impairment, tinnitus or an acoustic trauma, to prevent, reduce or treat the incidence of or severity of the hearing impairment with a prophylactically or therapeutically effective amount of a redox-active therapeutic, wherein said redox-active therapeutic is not Idebenone, Vitamin E, or Trolox (6-Hydroxy-2,5,7,8-tetramethylchroman-2-carbonic acid).

In another embodiment, the invention relates to a method for treating a patient having or prone to having an age-induced hearing impairment with a prophylactically or therapeutically effective amount of a redox-active therapeutic.

In another embodiment, the invention relates to a method for treating a patient with an ototoxin-induced or inducible hearing impairment, to prevent, reduce or treat the incidence of or severity of the hearing impairment with a prophylactically or therapeutically effective amount of a redox-active therapeutic.

In another embodiment, the invention relates to a method for treating a patient with an ototoxin-induced or inducible balance impairment, to prevent, reduce or treat the incidence of or severity of the balance impairment with a prophylactically or therapeutically effective amount of a redox-active therapeutic.

In another embodiment, the invention relates to a method of preventing or treating ototoxicity in a patient undergoing treatment with a pharmaceutical having an ototoxic-hearing impairment side effect, with a therapeutically effective amount of a redox-active therapeutic to prevent or treat the ototoxicity induced by the pharmaceuticals. In another embodiment, the invention relates to a method of treating a patient undergoing an antibiotic, an antimicrobial or an antifungal treatment with a pharmaceutical having an ototoxic-hearing impairment side effect, with a therapeutically effective amount of a redox-active therapeutic to treat the ototoxicity induced by said antibiotics or antimicrobials. In another embodiment, the invention relates to a method of treating a patient undergoing a treatment with an aminoglycoside antibiotic, having an ototoxic-hearing impairment side effect, with a therapeutically effective amount of a redox-active therapeutic such as Idebenone, CoQ10, vitamin E, or Trolox to treat the ototoxicity induced by said aminoglycosides.

In another embodiment, the invention relates to a method of treating a patient undergoing a treatment with an aminoglycoside antibiotic, such as gentamicin, having an ototoxic-hearing impairment side effect, with a therapeutically effective amount of a redox-active therapeutic such as a compound having a structure comprising a quinone moiety, to treat the ototoxicity induced by said aminoglycoside. Examples of such aminoglycoside antibiotics include but are not limited to gentamicins, streptomycins, kanamycins, tobramycins, and the like.

In another embodiment, the patient in need of a hearing impairment treatment is undergoing a treatment with an aminoglycoside antibiotic such as neomycin, amikacin, tobramycin, viomycin, gentamicin, sisomicin, netimicin, treptomycin, dibexacin, fortimcin and dihydrostreptomycin.

In another embodiment, the invention relates to a method of treating a patient having a neurotoxin induced hearing impairment with a therapeutically effective amount of a redox-active therapeutic. Examples of neurotoxins are glutamates and aspartates.

In another embodiment, the invention relates to a method of treating a patient with hearing impairments resulting from the administration of quinine or its synthetic substitutes with a therapeutically effective amount of a redox-active therapeutic.

In another embodiment, the invention relates to a method of treating a patient with hearing impairments resulting from the administration of diuretics, for example furosemide, ethacrynic acid and mercurials, with a therapeutically effective amount of a redox-active therapeutic.

In another embodiment, the invention relates to a method of treating a patient with hearing impairments resulting from the administration of anti-neoplastics, such as platinum-containing antineoplastic agents, with a therapeutically effective amount of a redox-active therapeutic. Examples of anti-neoplastic drugs are cisplatin or cisplatin-like compounds.

In another embodiment, the invention relates to a method of treating a patient with hearing impairments resulting from the administration of salicylate, i.e. aspirin, or salicylate-like compounds, with a therapeutically effective amount of a redox-active therapeutic.

In another embodiment, the invention relates to a method of treating a patient who cannot detect small changes in tone intensity, with a therapeutically effective amount of a redox-active therapeutic.

In another embodiment, the invention relates to a method of treating a patient who cannot continue to perceive a constant tone above the threshold of hearing, with a therapeutically effective amount of a redox-active therapeutic.

In another embodiment, the invention relates to a method for treating a patient with tinnitus or ringing of the ears, to prevent, reduce or treat the incidence of or severity of the tinnitus or ringing of the ears with a prophylactically or therapeutically effective amount of a redox-active therapeutic

In another embodiment, the invention relates to a method of treating damage to spiral ganglion neurons.

In another embodiment, the invention relates to a method for treating a patient with an ototoxin-induced or inducible balance impairment, to prevent, reduce or treat the incidence of or severity of the balance impairment with a prophylactically or therapeutically effective amount of a redox-active therapeutic.

In another embodiment, the invention relates to a method for treating a patient with an aminoglycoside antibiotic induced or inducible balance impairment, to prevent, reduce or treat the incidence of or severity of the balance impairment with a prophylactically or therapeutically effective amount of a redox-active therapeutic.

In another embodiment, the invention relates to a method for treating a patient with gentamicin induced or inducible balance impairment, to prevent, reduce or treat the incidence of or severity of the balance impairment with a prophylactically or therapeutically effective amount of a redox-active therapeutic.

In another embodiment, the invention relates to a method for treating a patient with an anti-neoplastic induced or inducible balance impairment, to prevent, reduce or treat the incidence of or severity of the balance impairment with a prophylactically or therapeutically effective amount of a redox-active therapeutic. In some embodiments the anti-neoplastic drug is cisplatin or a cisplatin-like compound.

In another embodiment, the invention relates to a method for treating a patient with a loop diuretic induced or inducible balance impairment, to prevent, reduce or treat the incidence of or severity of the balance impairment with a prophylactically or therapeutically effective amount of a redox-active therapeutic.

In another embodiment, the invention relates to a method for treating a patient with a neurotoxin induced or inducible balance impairment, to prevent, reduce or treat the incidence of or severity of the balance impairment with a prophylactically or therapeutically effective amount of a redox-active therapeutic. In some embodiments the neurotoxin is aspartate or glutamate.

In another embodiment, the invention relates to a composition comprising a medicament known to have an ototoxic-hearing or balance impairment side-effect in combination with a redox-active therapeutic as described herein, for administration to a patient in need of such treatment. In some embodiments, the compositions comprise an ototoxic medicament and a redox-active therapeutic of Formula I, Formula II, Formula III, Formula IV, Formula V or Formula VI. In some embodiments, the compositions comprise an ototoxic medicament and a redox-active therapeutics selected from alpha tocotrienol, beta tocotrienol, gamma tocotrienol or mixtures thereof. In other embodiments, the compositions comprise an ototoxic medicament and a naturally occurring plant extract comprising tocopherols and tocotrienols, such as a palm oil extract. In some embodiments, the compositions comprise an ototoxic medicament and redox-active therapeutics such as Idebenone, CoQ-10 and derivatives thereof. Other examples include compositions comprising an ototoxic medicament and vitamin E or/and Trolox. Other examples include compositions comprising an ototoxic medicament and redox-active compounds having a chemical structure with a quinone moiety as defined herein.

In other embodiments the invention relates to a composition including a combination of anti-neoplastic drugs such as cisplatin or a cisplatin-like compounds and a redox-active therapeutic as described herein.

In other embodiments the invention relates to a composition including a combination of aminoglycoside antibiotics such as gentamicins, streptomycins, kanamycins, or tobramycins and a redox-active therapeutic as described herein

In other embodiments, the invention relates to a composition including a combination of a neurotoxin drug such as aspartate or glutamate and a redox-active therapeutic as described herein.

In another embodiment, the invention relates to a composition comprising a medicament known to have an ototoxic-hearing or balance impairment side-effect in combination with two or more redox-active therapeutics, said composition being for administration to a patient in need of such treatment.

In another embodiment, the invention relates to a composition comprising a medicament known to have an ototoxic hearing or balance impairment side-effect in combination with a redox-active therapeutic and an additional antioxidant or a spin-trapping agent. Examples of antioxidants include but are not limited to allopurinol, glutathione, methionine, carnitine, N-acetyl cysteine, and ebselen

For all of the compounds and methods described above, the quinone form can also be used in its reduced (hydroquinone) form when desired. Likewise, the hydroquinone form can also be used in its oxidized (quinone) form when desired.

### MODES FOR CARRYING OUT THE INVENTION

The invention embraces compositions and methods for prophylactic and therapeutic treatment of hearing impairments, particularly for the treatment of ototoxin-induced hearing impairments involving neuronal damage, loss or degeneration of neurons in a patient, or for the prevention of toxic side effects of ototoxic medications, by administration of redox-active therapeutics. In some embodiments, the present invention relates to the use of redox-active therapeutics comprising a quinone core structure or its reduced (hydroquinone) form structure.

Additionally the invention also addresses compositions and methods for prophylactic and therapeutic treatment of balance impairments, particularly for the treatment of ototoxin-induced balance impairments involving neuronal damage, loss or degeneration of neurons in a patient, or for the prevention of toxic side effects of ototoxic medications, by administration of redox-active therapeutics. In some embodiments, the present invention relates to the use of redox-active therapeutics comprising a quinone core structure or its reduced (hydroquinone) form structure.

By “subject,” “individual,” or “patient” is meant an individual organism, preferably a vertebrate, more preferably a mammal, including humans, domestic, and farm animals, and zoo, sports, or pet animals, such as dogs, horses, cats, sheep, pigs, cows, etc. The preferred mammal herein is a human. The methods of the present invention are thus applicable to both human therapy and veterinary applications.

“Treating” a disease with the compounds and methods discussed herein is defined as administering one or more of the compounds discussed herein, with or without additional therapeutic agents, in order to reduce, eliminate or reverse either the disease or one or more symptoms of the disease, or to retard the progression of the disease or of one or more symptoms of the disease, or to reduce the severity of the disease or of one or more symptoms of the disease. “Suppression” of a disease with the compounds and methods discussed herein is defined as administering one or more of the compounds discussed herein, with or without additional therapeutic agents, in order to suppress the clinical manifestation of the disease, or to suppress the manifestation of adverse symptoms of the disease. The distinction between treatment and suppression is that treatment occurs after adverse symptoms of the disease are manifest in a subject, while suppression occurs before adverse symptoms of the disease are manifest in a subject. Suppression may be partial, substantially total, or total. The compounds and methods of the invention can be administered to asymptomatic patients at risk of developing the clinical symptoms of the disease, in order to suppress the appearance of any adverse symptoms.

Such treatment is expected to allow hair cells and/or auditory neurons to tolerate intermittent insults from either environmental noise trauma or treatment with ototoxins, and to slow down, prevent or reverse the progressive degeneration of the auditory neurons and hair cells which is responsible for hearing loss in pathological conditions such as presbycusis (age-related hearing loss), inherited sensorineural degeneration, and post-idiopathic hearing losses and to preserve the functional integrity of the inner ear. Such treatment will also support the auditory neurons for better and longer performance of cochlear implants.

“Treatment” refers to both therapeutic treatment and prophylactic or preventative measures, wherein the object is to prevent or slow down (lessen) neuron-damage-related hearing impairment, preferably ototoxin-induced or inducible. Those in need of treatment include those already experiencing a hearing impairment, those prone to having the impairment, and those in which the impairments are to be prevented. The hearing impairments are due to neuronal damage, wherein the damage is caused by infections, mechanical injury, loud sounds, aging, or chemical-induced ototoxicity, wherein ototoxins include therapeutic drugs including antibiotics, antimicrobials, antifungals, anti-neoplastic agents, salicylates, quinines, contaminants in foods or medicaments, and environmental or industrial pollutants. Typically, treatment is performed to prevent or reduce ototoxicity, especially resulting from or expected to result from administration of therapeutic drugs. The treatment may be performed with a therapeutically effective composition given immediately after the exposure to prevent or reduce the ototoxic effect, or prior to or concomitantly with the ototoxic pharmaceutical or the exposure to the ototoxin.

“Balance impairment” refers to a neurologic disorder, oto-neurological, in which the patient displays, complains of, or is diagnosed to have known diagnostic symptoms of a balance disorder, including ataxic gait, inability to stand on one leg, or inability to walk heel-to-toe, inability to tandem walk, and dizziness or vertigo that are neurologically related. During vertigo the patient may experience a subjective impression of movement in space (subjective vertigo) or of objects moving in space (objective vertigo) usually with a loss of equilibrium. These impairments of interest to the present invention are those typically associated with damage to neurons, and possibly hair cells, of the vestibular system related to the 8th cranial nerve. Particularly affected may be neurons of the vestibule, semicircular canal, 8th nerve, vestibular neurons of the brainstem and their temporal lobe connections, and more particularly the organ of Corti.

“Ototoxic agent” refers to a substance that through its chemical action injures, impairs, or inhibits the activity of a component of the nervous system related to hearing or balance, to in turn impair hearing or balance. The list of ototoxic agents that cause hearing or balance impairments includes, but is not limited to, neoplastic agents such as vincristine, vinblastine, cisplatin, taxol, or dideoxy-compounds, e.g., dideoxyinosine; alcohol; metals; industrial toxins involved in occupational or environmental exposure, including toluene, xylene, etc.; contaminants of food or medicaments; or over-doses of vitamins or therapeutic drugs, e.g., antibiotics such as penicillin, aminoglycosides, polypeptide antibiotics, or chloramphenicol, or large doses of vitamins A, D, or B6, salicylates, quinines and synthetic quinine-like compounds, and loop diuretics including furosemide, ethocrynic acid. By “exposure to an ototoxic agent” is meant that the ototoxic agent is made available to, or comes into contact with, a mammal. Exposure to an ototoxic agent can occur by direct administration, e.g., by ingestion or administration of a food, medicament, or therapeutic agent, e.g., a chemotherapeutic agent, by accidental contamination, or by environmental exposure, e.g., aerial or aqueous exposure.

“Aminoglycoside antibiotic” refers to a broad class of amino sugar containing antibiotics well known in the art. The aminoglycoside agents described in the literature which are useful in the methods of the present invention include, but are not limited to, amikacin (BB-K8), butirosin, geneticin, gentamicin, kanamycin, lividomycin, neomycin, paromomycin, hybrimycin, propikacin (UK 31214), ribostamycin, seldomycin, trehalosamine, α-D-mannosyl-α-D-glucosaminide, apramycin, bluensomycin, netromycin, streptomycin, sisomicin, destomycin, antibiotic A-396-I, dibekacin, kasugamycin, fortimicin, netilmicin, hygromycin, and tobramycin, and derivatives, analogs or variants thereof. Also useful in the methods of the invention are ototoxic glycopeptide antibiotics such as vancomycin, and ototoxic macrolide antibiotics such as erythromycin.

“Platinum-containing antineoplastic agents” refers to a broad class of water-soluble, platinum coordination compounds well known in the art, typically having anti-tumor activity. The platinum-containing antineoplastic agents described in the literature which are useful in the methods of the present invention include, but are not limited to, cis-diaminedichloro-platinum(II) (cisplatin), trans-diaminedichloro-platinum(II), cis-diamine-diaquaplatinum(II)-ion, cis-diaminedichloroplatinum(II)-ion, chloro(diethylenetriamine)-platinum(II) chloride, dichloro(ethylenediamine)-platinum(II), diamine(1,1-cyclobutanedicarboxylato)-platinum(II) (carboplatin), spiroplatin, dichlorotrans-dihydroxybisisopropolamine platinum IV (iproplatin), diamine(2-ethylmalonato)platinum(II), ethylenediamine-malonatoplatinum(II), aqua(1,2-diaminodiclohexane)-sulfatoplatinum(II), (1,2-diaminocyclohexane)malonato-platinum(II), (4-carboxyphthalato)(1,2-diaminocyclo-hexane)-platinum(II), (1,2-diaminocyclohexane)-(isocitrato)platinum(II), (1,2-diaminocyclohexane)-cis(pyruvato)platinum(II), and (1,2-diaminocyclohexane)-oxalatoplatinum(II).

A “therapeutically effective amount” of a compound is an amount of the compound, which, when administered to a subject, is sufficient to reduce or eliminate either a disease or one or more symptoms of a disease, or to retard or reverse the progression of a disease or of one or more symptoms of a disease, or to reduce the severity of a disease or of one or more symptoms of a disease, or to suppress the clinical manifestation of a disease, or to suppress the manifestation of adverse symptoms of a disease.

“(C₁-C₆)-alkyl” is intended to embrace a saturated linear, branched, cyclic, or a combination of linear and/or branched and/or cyclic hydrocarbon chain and/or ring of 1 to 6 carbon atoms. Examples of “(C₁-C₆)-alkyl” are methyl, ethyl, n-propyl, isopropyl, cyclopropyl, n-butyl, isobutyl, sec-butyl, t-butyl, cyclobutyl, cyclopropyl-methyl, methyl-cyclopropyl, pentyl where the point of attachment of the pentyl group to the remainder of the molecule can be at any location on the pentyl fragment, cyclopentyl, hexyl where the point of attachment of the hexyl group to the remainder of the molecule can be at any location on the hexyl fragment, and cyclohexyl. This term includes mono and divalent hydrocarbon chains, i.e. (C₁-C₆)-alkyl and (C₁-C₆)-alkylene chains of 1 to 6 carbon atoms.

(C₁-C₆)-alkylene is intended to embrace a divalent saturated linear, branched, cyclic, or a combination of linear and/or branched and/or cyclic hydrocarbon chain and/or ring of 1 to 6 carbon atoms.

“(C₀-C₆)-alkyl” is intended to embrace a saturated linear, branched, cyclic, or a combination of linear and/or branched and/or cyclic hydrocarbon chain and/or ring of 1 to 6 carbon atoms, as described above for (C₁-C₆)-alkyl, or where the alkyl group is absent; if the absence of the alkyl group results in an open valence, as in —C(═O)—C₀ alkyl, then C₀ alkyl represents a hydrogen atom. This term includes mono and divalent hydrocarbon chains, i.e. (C₁-C₆)-alkyl and (C₁-C₆)-alkylene chains of 1 to 6 carbon atoms.

“(C₁-C₆)-haloalkyl” is intended to embrace any (C₁-C₆)-alkyl substituent having at least one halogen substituent; the halogen can be attached via any valence on the (C₁-C₆)-alkyl group. One subset of (C₁-C₆)-haloalkyl is —CF₃, —CCl₃, —CBr₃, and —Cl₃. Another subset of (C₁-C₆)-haloalkyl is —CHF₂, —CHCl₂, —CHBr₂, and —CHI₂. Another subset of (C₁-C₆)-haloalkyl is —CH₂F, —CH₂Cl, —CH₂Br, and —CH₂I. Another subset of (C₁-C₆)-haloalkyl is the subset of (C₁-C₆)-perhaloalkyls where all available valences are replaced by halogens. Another subset of (C₁-C₆)-haloalkyl is the subset of (C₁-C₆)-perfluoroalkyl; where all available valences are replaced by fluorines. Another subset of (C₁-C₆)-haloalkyl is the subset of (C₁-C₆)-perchloroalkyl; that is, (C₁-C₆)-alkyl with all available valences replaced by chlorines.

The term “aryl” is intended to embrace an aromatic cyclic hydrocarbon group of from 6 to 20 carbon atoms having a single ring (e.g., phenyl) or multiple condensed (fused) rings (e.g., naphthyl or anthryl).

The terms “heterocycle”, “heterocyclic”, “heterocyclo”, and “heterocyclyl” is intended to encompass a monovalent, saturated, partially unsaturated, or unsaturated carbocyclic radical having one or more rings incorporating one, two, three or four heteroatoms within the ring (chosen from nitrogen, oxygen, and/or sulfur). Examples of saturated heterocycles include morpholine, piperidine, piperazine, thiazolidine, pyrazolidine, pyrazoline, imidazolidine, pyrrolidine, tetrahydropyran, tetrahydrofuran, quinuclidine, and the like. This term also includes heteroaryls as defined below.

The terms “heteroaryl”, is intended to encompass a monovalent aromatic, carbocyclic radical having one or more rings incorporating one, two, three or four heteroatoms within the ring (chosen from nitrogen, oxygen, and/or sulfur). Examples of heteroaryl include pyridine, pyrazine, imidazoline, thiazole, isothiazole, pyrazine, triazine, pyrimidine, pyridazine, pyrazole, thiophene, pyrrole, pyran, furan, indole, quinoline, quinazoline, benzimidazole, benzothiophene, benzofuran, benzoxazole, benzothiazole, benzotriazole, imidazo-pyridines, pyrazolo-pyridines, pyrazolo-pyrazine, acridine, carbazole, and the like.

An effective amount of redox-active therapeutic(s) to be employed therapeutically will depend, for example, upon the therapeutic objectives, the route of administration, the species of the patient, and the condition of the patient. Accordingly, it will be necessary for the therapist to titer the dosage and modify the route of administration as required to obtain the optimal therapeutic effect. As is known in the art, adjustments for age as well as the body weight, general health, sex, diet, time of administration, drug interaction and the severity of the disease may be necessary, and will be ascertainable with routine experimentation by those skilled in the art. A typical daily dosage of redox-active therapeutic used alone might range from about 1 μg/kg to up to 100 mg/kg of patient body weight or more per day, depending on the factors mentioned above, preferably about 10 μg/kg/day to 10 mg/kg/day. Typically, the clinician will administer redox-active therapeutic until a dosage is reached that repairs, maintains, and, optimally, reestablishes neuron function to relieve the hearing impairment. Generally, the redox-active therapeutic is formulated and delivered to the target site at a dosage capable of establishing at the site an agonist level greater than about 0.1 ng/ml, more typically from about 0.1 ng/ml to 5 mg/ml, preferably from about 1 ng/ml to 2000 ng/ml.

The redox-active therapeutic(s) optionally may be combined with or administered in concert with ototoxic pharmaceutical drugs. Initially the drugs are administered in conventional therapies known for the ototoxic pharmaceutical. Adjustments to the therapies are at the discretion of the skilled therapist to titrate dosages and conditions that decrease ototoxicity-related hearing while maintaining, and preferably improving, treatment outcomes with the ototoxic pharmaceutical drug.

If redox-active therapeutics are administered in concert with ototoxic pharmaceutical drugs, they need not be administered by the same route, nor in the same formulation. However, they can be combined into one formulation as desired.

Some pharmaceutical compositions comprise an effective ototoxicity-inhibiting amount of redox-active therapeutic as described herein, a therapeutically effective amount of the ototoxic pharmaceutical drug, such as an aminoglycoside antibiotic, or and anti-neoplastic agent such as cisplatin, and optionally a pharmaceutically acceptable carrier in concert with ototoxic pharmaceutical drugs and/or vehicle which would be familiar to one skilled in the pharmaceutical arts. The actual amounts of ototoxic pharmaceutical drug employed will range from those given in standard references for prescription drugs, e.g. “Physicians Desk Reference” (1995), “Drug Evaluations” AMA, 6th Edition (1986); to amounts somewhat larger since the ototoxicity potential is reduced in these compositions.

The effective amounts of such agents, if employed, will be at the physician's or veterinarian's discretion. Dosage administration and adjustment is done to achieve the best management of hearing or balance (and when used in conjunction with an ototoxic pharmaceutical drug, the indication for the ototoxic drug). The dose will additionally depend on such factors as the type of drug used and the specific patient being treated. Typically the amount employed will be the same dose as that used if the drug were to be administered without agonist; however, lower doses may be employed depending on such factors as the presence of side-effects, the condition being treated, the type of patient, and the type of agonist and drug, provided the total amount of agents provides an effective dose for the condition being treated. For example, a test dose may be 5 mg, which is then ramped up to 10-20 mg per day, once a day, to 25 mg twice per day (BID) or three times per day (TID), and may be titrated to 50 mg BID or TID as the patient tolerates it. Tolerance level is estimated by determining whether decrease in hearing impairment is accompanied by signs of observed side-effects. A discussion of the dosage, administration, indications and contraindications associated with ototoxic pharmaceuticals optionally used with the redoxactive therapeutics in the methods of the invention can be found in the Physicians Desk Reference, Medical Economics Data Production Co., Montvale, N.J. (1995).

The effectiveness of treating hearing impairments with the methods of the invention can be evaluated by the following signs of recovery, including recovery of normal hearing function or balance function, which can be assessed by known diagnostic techniques including those discussed herein, and normalization of nerve conduction velocity, which is assessed electro-physiologically.

“Redox-active therapeutics” refers to therapeutics comprising a moiety having the property of giving up electrons to a suitable oxidizing agent or taking up electrons from a suitable reducing agent. For the purposes of the present invention, the preferred moieties comprise but are not limited to a quinone core structure or a tocotrienol core structure. Some examples of redox active therapeutics are CoQ-10, Idebenone, Ubiquinone, Mitoquinone (Mito-Q) and their derivatives. Further examples of redox active therapeutics are provided in co-assigned US Pat. publications No. 2006/0281809, 2007/0072943, 2007/0225261, and U.S. Provisional Pat. applications No. 61/002,126, 61/002,127, 61/010,409 and 61/010,387, incorporated herein by reference. Other examples of therapeutics having chemical structure comprising a quinone moiety included in but not limiting the invention are AA-861 (Takeda); E-6700 and E-3300 (Eisai); Seratrodast™ (Abbott); CV-6504 (Takeda); BN-8265 and IRC-083864 (SCRAS); and HU-331(Hebrew University). For the purposes of the present invention the quinone moiety in the chemical structure of the redox-active therapeutic may be isolated or embedded in a larger structure such as but no limited to a naphthaquinone, anthraquinone or a larger molecule such as Mitomycin. For the purpose of the present invention the term includes pro-drugs of the redox-active compounds as defined herein.

For the purpose of the invention the redox therapeutic can be a naturally occurring phytonutrient or a plant extract with redox properties. The redox-active therapeutic may be a natural mixture of tocopherols and tocotrienols extracted from palm oil or cereal grains (such as oat, barley, and rye, rice bran). The redox-active therapeutic may be a mixture of tocopherols and tocotrienol sold by Carotech as Tocomin® or Tocomin® SupraBio™ a series of products containing natural occurring mixture of tocotrienols and tocopherol extracted and concentrated from virgin crude palm oil/palm fruits (Elaeis guineensis).

“Essentially pure” tocotrienol refers to a tocotrienol of at least 60% purity; or at least 70% purity; or at least 80% purity; or at least 90% purity; or at least 95% purity; or at least 99% purity.

### Examples of Redox-Active Therapeutic Compounds.

Some redox active therapeutic compounds used in the treatment of hearing impairments are compounds of Formula I:

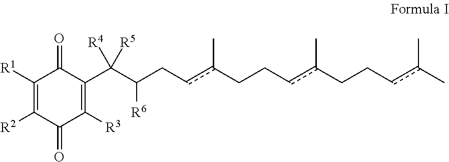

wherein,
the bonds indicated with a dashed line can independently be single or double,
R¹, R², and R³ are independently selected from H, (C₁-C₄)-alkyl, (C₁-C₄)-haloalkyl, CN, F, Cl, Br, and I; and
R⁴ is independently selected from hydroxy and (C₁-C₄)-alkyl, R⁵ is independently selected from (C₁-C₄)-alkyl, and R⁶ is hydrogen; or
R⁴ is alkyl, and R⁵ and R⁶ are hydrogen; or
R⁴ is alkyl, and R⁵ and R⁶ together form a double bond;
and salts, stereoisomers, mixtures of stereoisomers, prodrugs, metabolites, sovates and hydrates thereof.

Some other redox active therapeutic compounds used in the treatment of hearing impairments are compounds of Formula II:

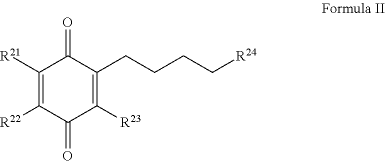

wherein,
R²¹, R²², and R²³ are independently selected from H, (C₁-C₄)-alkyl, (C₁-C₄)-haloalkyl, CN, F, Cl, Br, and I;
R²⁴ is independently selected from (C₁-C₂₀)-alkyl, (C₁-C₂₀)-alkenyl; (C₁-C₂₀)-alkynyl and (C₁-C₂₀) containing at least one double bond and at least one triple bond,
   and all salts, stereoisomers, mixtures of stereoisomers, prodrugs, metabolites, solvates and hydrates thereof.

Some other redox active therapeutic compounds used in the treatment of hearing impairments are compounds of Formula III:

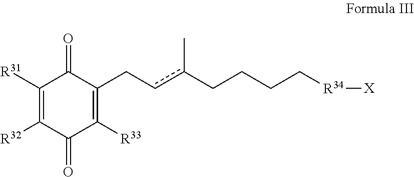

wherein,
the bond indicated with a dashed line can be single or double;
R³¹, R³², and R³³ are independently selected from the group consisting of H, (C₁-C₅)-alkyl, (C₁-C₅)-haloalkyl, (C₂-C₅)-alkenyl, (C₂-C₅)-haloalkenyl, (C₂-C₅)-alkynyl, —(C₁-C₅)-haloalkynyl, OR³⁵, SR³⁵, CN, F, Cl, Br, I, N₃, and NR³⁵R³⁶; where R³⁵ and R³⁶ are independently selected from the group consisting of H, (C₁-C₅)-alkyl, (C₃-C₅)-cycloalkyl, (C₁-C₅)-haloalkyl, aryl, heteroaryl, —(C═O)—(C₁-C₈)-alkyl, and —(C═O—(C₀-C₈)-alkyl-(C₆-C₁₀)-aryl-(C₀-C₈)-alkyl, or where R³⁵ and R³⁶ selected from these groups are combined to form a ring;
   R³⁴ represents a linear or branched group containing 1 to 32 carbon atoms and any number of single, double, or triple bonds in any chemically possible combination;
   X is selected from the group consisting of H, F, Cl, Br, I, CN, —N₃, —NR³⁷R³⁸, and OR³⁹; where R³⁷ and R³⁸ are independently selected from H, (C₁-C₈)-alkyl or (C₁-C₈)-haloalkyl, —(C═O)—(C₁-C₈)-alkyl, or where either one of R³⁷ and R³⁸ are independently selected from the group consisting of —(C═O)—(C₁-C₈)-haloalkyl; —(C═O)—NH₂; —(C═O)—NH(C₁-C₈)-alkyl; —(C═O)—NH(C₁-C₈)-haloalkyl; —(C═O)—NR³⁰¹R³⁰², where R³⁰¹ and R³⁰² together with the nitrogen atom to which they are attached combine to form a 3- to 8-membered ring, and where another group selected from —NH—, —N((C₁-C₄)-alkyl)-, —O—, or —S— can be optionally incorporated in the ring formed by R³⁰¹ and R³⁰² and the nitrogen atom to which they are attached; —(C═O)—O—(C₁-C₈)-alkyl, —(C═O)—O—(C₁-C₈)-haloalkyl, —S(O)₂—(C₁-C₈)-alkyl, —S(O)₂-aryl, and —S(O)₂-aralkyl, and where the other of R³⁷ or R³⁸ is H, (C₁-C₈)-alkyl or (C₁-C₈)-haloalkyl or where R³⁷ and R³⁸ selected from these groups together with the nitrogen atom to which they are attached combine to form a 3- to 8-membered ring, and where another group selected from —NH—, —N((C₁-C₄)-alkyl)-, —O—, or —S— can be optionally incorporated in the ring formed by R³⁷ and R³⁸ and the nitrogen atom to which they are attached; where R³⁹ is independently selected from H, —(C₁-C₈)-alkyl or (C₁-C₈)-haloalkyl, —(C═O)—(C₁-C₈)-alkyl, —(C═O)—(C₁-C₈)-haloalkyl, —(C═O)—NH₂, —(C═O)—NH—(C₁-C₈)-alkyl, —(C═O)—NH(C₁-C₈)-haloalkyl, —(C═O)—NR³⁰¹R³⁰² where R³⁰¹ and R³⁰² together with the nitrogen atom to which they are attached combine to form a 3- to 8-membered ring, and where another group selected from —NH—, —N((C₁-C₄)-alkyl)-, —O—, or —S— can be optionally incorporated in the ring formed by R³⁰¹ and R³⁰² and the nitrogen atom to which they are attached, —(C═O)—O—(C₁-C₈)-alkyl, —(C═O)—O—(C₁-C₈)-haloalkyl, —S(O)₂—(C₁-C₈)-alkyl, —S(O)₂-aryl, and —S(O)₂-aralkyl; with the proviso that when both of R³¹ and R³² are —OCH₃ and R³³ is —CH₃, then X is not —H or —OH;
or any stereoisomer, mixture of stereoisomers, prodrug, metabolite, salt, phosphate-substituted form, sulfate-substituted form, phosphate/sulfate substituted form, crystalline form, non-crystalline form, hydrate, or solvate thereof.

Some other redox active therapeutic compounds used in the treatment of hearing impairments are compounds of Formula IV:

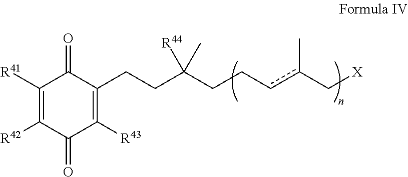

wherein,
n is an integer from 0 to 9 inclusive, and each unit can be the same or different; the bond(s) indicated by a dashed line can independently of each other be single or double bonds;
R⁴¹, R⁴², and R⁴³ are independently selected from the group consisting of H, (C₁-C₅)-alkyl, (C₁-C₅)-haloalkyl, (C₂-C₅)-alkenyl, (C₂-C₅)-haloalkenyl, (C₂-C₅) alkynyl, (C₂-C₅)-haloalkynyl, —OR⁴⁵, —SR⁴⁵, CN, F, Cl, Br, I, N₃, and —NR⁴⁵R⁴⁶; where R⁴⁵ and R⁴⁶ are independently selected from the group consisting of H, (C₁-C₈)-alkyl, (C₃-C₆)-cycloalkyl, (C₁-C₅)-haloalkyl, aryl, heteroaryl, —(C═O)—(C₁-C₈)-alkyl, and —(C═O)—(C₀-C₈)-alkyl-(C₆-C₁₀)aryl-(C₀-C₄)alkyl, or where R⁴⁵ and R⁴⁶ selected from these groups are combined to form a ring;
R⁴⁴ is selected from the group consisting of H, —OR⁴⁵, —SR⁴⁵, F, Cl, Br, I, and —NR⁴⁵R⁴⁶;
X is selected from the group consisting of H, —NR⁴⁷R⁴⁸, —OR⁴⁹ and —(CH₂)₂C(CH₃)₂OH;
R⁴⁷ and R⁴⁸ are independently selected from H, —(C₁-C₈)-alkyl or (C₁-C₈)haloalkyl, —(C═O)—(C₁-C₈)-alkyl, or where either one of R⁴⁷ and R⁴⁸ are independently selected from the group consisting of —(C═O)—(C₁-C₈)-haloalkyl, —(C═O)—NH₂, —(C═O)—(C₁-C₈)alkyl, —(C═O)—NH(C₁-C₈)-haloalkyl, —(C═O)—NR⁴⁰¹R⁴⁰² where R⁴⁰¹ and R⁴⁰² together with the nitrogen atom to which they are attached combine to form a 3- to 8-membered ring, and where another group selected from —NH—, —N((C₁-C₄)alkyl)-, —O—, or —S— can be optionally incorporated in the ring formed by R⁴⁰¹ and R⁴⁰² and the nitrogen atom to which they are attached; —(C═O)—O—(C₁-C₈)alkyl, —(C═O)—O(C₁-C₈)-haloalkyl, —S(O)₂—(C₀-C₈)-alkyl, —S(O)₂-aryl, and —S(O)₂-aralkyl, and where the other of R⁴⁷ or R⁴⁸ is H, (C₁-C₈)-alkyl or (C₁-C₈)-haloalkyl or where R⁴⁷ and R⁴⁸ selected from these groups are combined to form a ring, or where R⁴⁷ and R⁴⁸ together with the nitrogen atom to which they are attached combine to form a 3- to 8-membered ring, and where another group selected from —NH—, —N((C₁-C₄)-alkyl)-, —O—, or —S— can be optionally incorporated in the ring formed by R⁴⁷ and R⁴⁸ and the nitrogen atom to which they are attached; where R⁴⁹ is independently selected from H, (C₁-C₈)-alkyl or (C₁-C₈)-haloalkyl, —(C═O)—(C₁-C₈)-alkyl, —(C═O)—(C₁-C₈)haloalkyl, —(C═O)—NH₂, —(C═O)——(C═O)—NH(C₁-C₈)-haloalkyl, —(C═O)—NR⁴⁰¹R⁴⁰² where R⁴⁰¹ and R⁴⁰² together with the nitrogen atom to which they are attached combine to form a 3- to 8-membered ring, and where another group selected from —NH—, —N((C₁-C₄)-alkyl)-, —O—, or —S— can be optionally incorporated in the ring formed by R⁴⁰¹ and R⁴⁰² and the nitrogen atom to which they are attached;—(C═O)—(C₁-C₈)-alkyl, —(C═O)—O(C₁-C₈)-haloalkyl, —S(O)₂(C₁-C₈)-alkyl, —S(O)₂-aryl, and —S(O)₂-aralkyl; with the provisos that when n=3 and if R⁴⁴ is —H or —OH, then X is not —H, and that when R⁴¹ and R⁴² are OCH₃ and R⁴³ is CH₃, then either R⁴⁴ is neither H nor —OH, or X is neither H nor —OH nor —(CH2)₂C(CH₃)₂OH;
or any stereoisomer, mixture of stereoisomers, prodrug, metabolite, salt, phosphate-substituted form, sulfate-substituted form, phosphate/sulfate substituted form, crystalline form, non-crystalline form, hydrate, or solvate thereof.

Some other redox active therapeutic compounds used in the treatment of hearing impairments are compounds of Formula V:

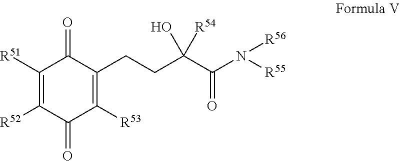

wherein,
R⁵¹, R⁵², and R⁵³ are independently selected from hydrogen and (C₁-C₆)-alkyl;
R⁵⁴ is (C₁-C₆)-alkyl;
R⁵⁵and R⁵⁶ are independently selected from hydrogen, hydroxy, alkoxy, (C₁-C₄₀)-alkyl, (C₁-C₄₀)-alkenyl, (C₁-C₄₀)-alkynyl, and aryl, with the proviso that only one of R⁵⁵ and R⁵⁶ is hydroxy; where the alkyl, alkenyl, alkynyl or aryl groups may optionally be substituted with
   —OR⁵⁰¹, —S(O)₀₋₂R⁵⁰¹, —CN, —F, —Cl, —Br, —I, —NR⁵⁰¹R⁵⁰², oxo, (C₃-C₆)-cycloalkyl, aryl, aryl-(C₁-C₆)-alkyl, heteroaryl, heterocyclyl, —C(═O)—R⁵⁰³, —C(═O)—(C₀-C₆)-alkyl-aryl, —C(═O)—O—R⁵⁰³, —C(═O)—O—(C₀-C₆)-alkyl-aryl, —C(═O)—N—R⁵⁰³R⁵⁰⁴, C(═O)—N—(C₀-C₆)-alkyl-aryl, —N—C(═O)—R⁵⁰³, —N—C(═O)—(C₀-C₆)-alkyl-aryl; where the aryl, heteroaryl and heterocyclyl ring substituents may be further substituted with (C₁-C₆)-alkyl, (C₁-C₆)-haloalkyl, oxo, hydroxy, (C₁-C₆)-alkoxy, —C(═O)—(C₁-C₆)-alkyl and —C(═O)—O—(C₁-C₆)-alkyl; and where one of the carbons of the alkyl, alkenyl, or alkynyl groups may be replaced by a heteroatom selected from O, N or S; or
R⁵⁵ and R⁵⁶ together with the atom to which they are attached form a saturated or unsaturated 3-8 membered ring, optionally incorporating one or more additional heteroatoms independently selected from one, two, or three N, O, or S atoms, optionally substituted with oxo, —OR⁵⁰¹, —SR⁵⁰¹, —CN, —F, —Cl, —Br, —I, —NR⁵⁰¹R⁵⁰², (C₁-C₆)-alkyl, (C₁-C₆)-haloalkyl; hydroxy-(C₁-C₆)-alkyl, —C(═O)—H, —C(═O)—(C₁-C₆)-alkyl , —C(═O)-aryl, —C(═O)—OH, or —C(═O)—O—(C₁-C₆)-alkyl; or
R⁵⁵ and R⁵⁶ together with the nitrogen atom to which they are attached form a N,N′-disubstituted piperazine where the nitrogen substitution at the 4-position is a group identical to the substitution at the 1-position forming a compound of formula Va, where R⁵¹, R⁵², R⁵³, and R⁵⁴ are as defined above:

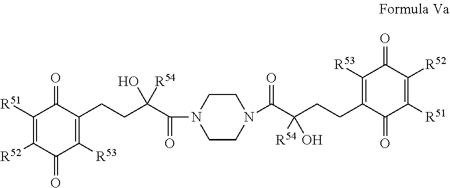

R⁵⁰¹ and R⁵⁰² are independently selected from the group consisting of hydrogen, (C₁-C₆)-alkyl, (C₁-C₆)-haloalkyl, aryl, aryl-(C₁-C₆)-alkyl, heteroaryl, heterocyclyl, —C(═O)—H, —C(═O)—(C₁-C₆)-alkyl, —C(═O)-aryl and —C(═O)—(C₁-C₆)-alkyl-aryl; and
R⁵⁰³ and R⁵⁰⁴ are selected from hydrogen and (C₁-C₆)-alkyl;
   and all salts, stereoisomers, mixtures of stereoisomers, prodrugs, metabolites, solvates, and hydrates thereof.

Some other redox active therapeutic compounds used in the treatment of hearing impairments are compounds of Formula VI:

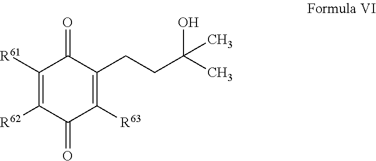

wherein,
R⁶¹ is aryl-(C₀-C₆)-alkyl- or heterocyclyl-(C₀-C₆)-alkyl-, wherein the aryl or heterocyclyl is optionally substituted with one or more substituents selected from (C₁-C₆)-alkyl, (C₂-C₆)-alkenyl, (C₂-C₆)-alkynyl, halogen, (C₁-C₆)-haloalkyl, hydroxy, (C₁-C₆)-alkoxy, CN, nitro, —C(═O)OR⁶⁴, —NR⁶⁵R⁶⁶, —C(═O)NR⁶⁵R⁶⁶, —SH, (C₁-C₆)-thioalkyl, and —C(═O)R⁶⁴; and wherein the (C₀-C₆)-alkyl group is optionally substituted with OH, —O—(C₁-C₄)-alkyl, —NH₂, —N ((C₁-C₄)-alkyl)₂, oxo or halogen; and
R⁶² and R⁶³ are independently selected from hydrogen, halogen, (C₁-C₆)-alkyl and (C₁-C₆)-alkoxy; or
R⁶³ is aryl-(C₀-C₆)-alkyl- or heterocyclyl-(C₀-C₆)-alkyl-, wherein the aryl or heterocyclyl is optionally substituted with one or more substituents selected from (C₁-C₆)-alkyl, (C₂-C₆)-alkenyl, (C₂-C₆)-alkynyl, halogen, (C₁-C₆)-haloalkyl-, hydroxy, (C₁-C₆)-alkoxy, CN, nitro, —C(═O)OR⁶⁴, —NR⁶⁵R⁶⁶, —C(═O)NR⁶⁵R⁶⁶, —SH, (C₁-C₆)-thioalkyl-, and —C(═O)R⁶⁴; and wherein the (C₀-C₆)-alkyl group is optionally substituted with OH, —O(C₁-C₄)-alkyl, —NH₂, —NH(C₁-C₄)-alkyl, —N ((C₁-C₄)-alkyl)₂, oxo or halogen; and
R⁶¹ and R⁶² are independently selected from hydrogen, halogen, (C₁-C₆)-alkyl, and (C₁-C₆)-alkoxy;
R⁶⁴ is hydrogen, (C₁-C₆)-alkyl, aryl, or aryl-(C₁-C₆)-alkyl-;
R⁶⁵ and R⁶⁶ are independently of each other hydroxy, (C₁-C₆)-alkoxy, (C₁-C₆)-alkyl, (C₂-C₆)-alkenyl, (C₂-C₆)-alkynyl, aryl, aryl-(C₁-C₆)-alkyl-, heterocyclyl, or heterocyclyl-(C₁-C₆)-alkyl-; wherein the alkyl, alkenyl, alkynyl, aryl and heterocyclyl groups can be further substituted with oxo, halogen, (C₁-C₆)-haloalkyl, hydroxy, (C₁-C₆)-alkoxy, or C(═O)OR⁶⁴;
   and all salts, stereoisomers, mixtures of stereoisomers, prodrugs, metabolites, solvates, and hydrates thereof.

Some examples of redox-active therapeutics described in co-assigned US publications 2006/0281809, 2007/0072943, and 2007/0225261 are:
alpha-tocopherol quinone (alternatively named as 2-(3-hydroxy-3,7,11,15-tetramethylhexadecyl)-3,5,6-trimethylcyclohexa-2,5-diene-1,4-dione);
2,3,5-trimethyl-6-(3,7,11,15-tetramethyl-hexadecyl)-[1,4]benzoquinone;
beta-tocopherol quinone;
gamma-tocopherol quinone;
alpha-tocotrienol quinone (alternatively named as 2-(3-hydroxy-3,7,11,15-tetramethyl-6,10,14-hexadecatrienyl)-3,5,6-trimethyl-2,5-cyclohexadiene-1,4-dione or 2-(3-hydroxy-3,7,11,15-tetramethyl-6,10,14-hexadecatrienyl)-3,5,6-trimethyl-p-benzoquinone, CAS Registry number 14101-66-7);
beta-tocotrienol quinone;
gamma-tocotrienol quinone;
2,3,5-trimethyl-6-(3,7,11,15-tetramethylhexadecyl)-2,5-cyclohexadiene-1,4-dione;
2,3,5-trimethyl-6-(3,7,11,15-tetramethyl-2,6,10,14-hexadecatetraenyl)-2,5-cyclohexadiene-1,4-dione;
2-butyl-3-(3-hydroxy-3,7,11,15-tetramethylhexadecyl)-5,6-dimethylcyclohexa-2,5-diene-1,4-dione;
2,3,5-trimethyl-6-(3,7,11,15-tetramethyl-hexadeca-2,6,10,14-tetraenyl)-[1,4]benzoquinone;
2-butyl-3-(3-hydroxy-3,7,11,15-tetramethyl-hexadecyl)-5,6-dimethyl-[1,4]benzoquinone;
2-(3-hydroxy-3,7,11,15-tetramethyl-hexadec y1)-5,6-dimethyl-3-propyl-[1,4]benzoquinone;
3-(3-hydroxy-3,7,11,15-tetramethyl-hexadecyl)-5-methyl-2-propyl-[1,4]benzoquinone;
2-(3-hydroxy-3,7,11,15-tetramethyl-hexadecyl)-3-isobutyl-5,6-dimethyl-[1,4]benzoquinone;
3-hydroxy-3,7,11,15-tetramethylhexadecyl -3,5,6-trimethyl-2,5-cyclohexadiene-1,4-dione;
2-hexyl-3,5,6-trimethyl-[1,4]benzoquinone;
2-octyl-3,5,6-trimethyl-[1,4]benzoquinone;
2-heptadeca-8,11-dienyl-3,5,6-trimethyl-[1,4]benzoquinone;
2-heptadec-8-enyl-3,5,6-trimethyl-[1,4]benzoquinone;
2-tert-butyl-3-hexyl-5,6-dimethyl-[1,4]benzoquinone;
2-heptadeca-8,11-dienyl-3,5-diisopropyl-6-methyl-[1,4]benzoquinone;
2-heptyl-3,5-diisopropyl-6-methyl-[1,4]benzoquinone;
2,3-dimethyl-5,6-bis-(3-methyl-butyl)-[1,4]benzoquinone;
2-(3-hydroxy-3-methyl-butyl)-5,6-dimethyl-3-(3-methyl-but-2-enyl)-[1,4]benzoquinone;
2-(3-hydroxy-3-methyl-butyl)-5,6-dimethyl-3-(3-methyl-butyl)-[1,4]benzoquinone;
2-(7-chloro-3-methylhept-2-enyl)-3,5,6-trimethylcyclohexa-2,5-diene-1,4-dione;
2-(6-chloro-3-methylhex-2-enyl)-3,5,6-trimethylcyclohexa-2,5 -diene-1,4-dione;
2-(6-iodo-3-methylhex-2-enyl)-3,5,6-trimethylcyclohexa-2,5-diene-1,4-dione;
2,3,5-trimethyl-6-(3-methylnon-2-enyl)-1,4-benzoquinone;
5-methyl-7-(2,4,5-trimethyl-3,6-dioxocyclohexa-1,4-dienyl)hept-5-enenitrile;
N-(5-methyl-7-(2,4,5-trimethyl-3,6-dioxocyclohexa-1,4-dienyl)hept-5-enyl)acetamide;
5-methyl-7-(2,4,5-trimethyl-3,6-dioxocyclohexa-1,4-dienyl)hept-5-enal;
2-(7-hydroxy-3-methylhept-2-enyl)-3,5,6-trimethylcyclohexa-2,5-diene-1,4-dione
2-tert-butyl-5,6-dimethyl-3-(3-methylnon-2-enyecyclohexa-2,5-diene-1,4-dione
2-(3,16-dihydroxy-3,7,11,15-tetramethylhexadeca-6,10,14-trienyl)-3,5,6-trimethylcyclohexa-2,5-diene-1,4-dione;
2-(16-amino-3-hydroxy-3,7,11,15-tetramethylhexadeca-6,10,14-trienyl)-3,5,6-trimethylcyclohexa-2,5-diene-1,4-dione;
2-(15-hydroxy-3,7,11,15-tetramethylhexadecyl)-3,5,6-trimethylcyclohexa-2,5-diene-1,4-dione;
2-(3-chloro-15-hydroxy-3,7,11,15-tetramethylhexadecyl)-3,5,6-trimethylcyclohexa-2,5-diene-1,4-dione
2-(3-chloro-15-hydroxy-3,7,11,15-tetramethylhexadeca-6,10-dienyl)-3,5,6-trimethylcyclohexa-2,5-diene-1,4-dione
2-(3-hydroxy-3-methylbutyl)-3-isopentyl-5,6-dimethylcyclohexa-2,5-diene-1,4-dione;
2,3-diisopentyl-5,6-dimethylcyclohexa-2,5-diene-1,4-dione
7-5-methyl-7-(2,4,7-trimethyl-3,6-dioxocyclohexa-1,4-dienyl)hept-5-enyl acetate; and
2-(7-hydroxy-3-methylhept-2-enyl)-3,5,6-trimethylcyclohexa-2,5-diene-1,4-dione;
   and all stereoisomers, mixture of stereoisomers, prodrugs, metabolites, salts, phosphate substituted form, crystalline form, non-crystalline form, hydrate or solvate thereof.

Some examples of redox-active therapeutics described in co-assigned U.S. provisional applications 61/002,126, 61/002,127, 61/010,409 and 61/010,387 are:
6,6′-(4,4′-(piperazine-1,4-diyl)bis(3-hydroxy-3-methyl-4-oxobutane-4,1-diyl))bis(2,3,5-trimethylcyclohexa-2,5-diene-1,4-dione);
2-hydroxy-N-(2-hydroxyethyl)-2-methyl-4-(2,4,5-trimethyl-3,6-dioxocyclohexa-1,4-dienyl)butanamide;
2-(3-hydroxy-3-methyl-4-oxo-4-(piperidin-1-yl)butyl)-3,5,6-trimethylcyclohexa-2,5-diene-1,4-dione;
N-hexyl-2-hydroxy-2-methyl-4-(2,4,5-trimethyl-3,6-dioxocyclohexa-1,4-dienyl)butanamide;
N-benzyl-2-hydroxy-2-methyl-4-(2,4,5-trimethyl-3,6-dioxocyclohexa-1,4-dienyl)butanamide;
N-(cyclopropylmethyl)-2-hydroxy-2-methyl-4-(2,4,5-trimethyl-3,6-dioxocyclohexa-1,4-dienyl)butanamide;
2-hydroxy-2-methyl-N-phenethyl-4-(2,4,5-trimethyl-3,6-dioxocyclohexa-1,4-dienyl)butanamide;
2-hydroxy-N-(3-hydroxypropyl)-2-methyl-4-(2,4,5-trimethyl-3,6-dioxocyclohexa-1,4-dienyl)butanamide;
N-cyclopropyl-2-hydroxy-2-methyl-4-(2,4,5-trimethyl-3,6-dioxocyclohexa-1,4-dienyl)butanamide;
2-(3-hydroxy-4-(4-hydroxypiperidin-1-yl)-3-methyl-4-oxobutyl)-3,5,6-trimethylcyclohexa-2,5-diene-1,4-dione;
2-hydroxy-2-methyl-4-(2,4,5-trimethyl-3,6-dioxocyclohexa-1,4-dienyl)butanamide;
2-hydroxy-N-(4-hydroxybutyl)-2-methyl-4-(2,4,5-trimethyl-3,6-dioxocyclohexa-1,4-dienyl)butanamide;
2-hydroxy-N-(5-hydroxypentyl)-2-methyl-4-(2,4,5-trimethyl-3,6-dioxocyclohexa-1,4-dienyl)butanamide;
2-hydroxy-N-(1-hydroxypropan-2-yl)-2-methyl-4-(2,4,5-trimethyl-3,6-dioxocyclohexa-1,4-dienyl)butanamide;
methyl 2-(2-hydroxy-2-methyl-4-(2,4,5-trimethyl-3,6-dioxocyclohexa-1,4-dienyl)butanamido)acetate;
N-(3-(1H-imidazol-1-yl)propyl)-2-hydroxy-2-methyl-4-(2,4,5-trimethyl-3,6-dioxocyclohexa-1,4-dienyl)butanamide;
2-hydroxy-N-(2-(2-hydroxyethoxy)ethyl)-2-methyl-4-(2,4,5-trimethyl-3,6-dioxocyclohexa-1,4-dienyl)butanamide;
2-hydroxy-2-methyl-N-(pyridin-2-ylmethyl)-4-(2,4,5-trimethyl-3,6-dioxocyclohexa-1,4-dienyl)butanamide;
2-hydroxy-2-methyl-N-(3-(2-oxopyrrolidin-1-yl)propyl)-4-(2,4,5-trimethyl-3,6-dioxocyclohexa-1,4-dienyl)butanamide;
2-hydroxy-N-(6-hydroxyhexyl)-2-methyl-4-(2,4,5-trimethyl-3,6-dioxocyclohexa-1,4-dienyl)butanamide;
2-(3-hydroxy-3-methyl-4-(4-methylpiperazin-1-yl)-4-oxobutyl)-3,5,6-trimethylcyclohexa-2,5-diene-1,4-dione;
2-(4-(4-benzylpiperazin-1-yl)-3-hydroxy-3-methyl-4-oxobutyl)-3,5,6-trimethylcyclohexa-2,5-diene-1,4-dione;
2-hydroxy-2-methyl-N-(3-morpholinopropyl)-4-(2,4,5-trimethyl-3,6-dioxocyclohexa-1,4-dienyl)butanamide;
2-hydroxy-N,N-bis(2-hydroxyethyl)-2-methyl-4-(2,4,5-trimethyl-3,6-dioxocyclohexa-1,4-dienyl)butanamide;
N-(2-(dimethylamino)ethyl)-2-hydroxy-2-methyl-4-(2,4,5-trimethyl-3,6-dioxocyclohexa-1,4-dienyl)butanamide;
2-hydroxy-N-(4-hydroxyphenethyl)-2-methyl-4-(2,4,5 -trimethyl-3,6-dioxocyclohexa-1,4-dienyl)butanamide;
N-(3-(dimethylamino)propyl)-2-hydroxy-2-methyl-4-(2,4,5 -trimethyl-3,6-dioxocyclohexa-1,4-dienyl)butanamide;
2-(4-(4-acetylpiperazin-1-yl)-3-hydroxy-3-methyl-4-oxobutyl)-3,5,6-trimethylcyclohexa-2,5-diene-1,4-dione;
2-(4-(azepan-1-yl)-3-hydroxy-3-methyl-4-oxobutyl)-3,5,6-trimethylcyclohexa-2,5-diene-1,4-dione;
2-(3-hydroxy-3-methyl-4-oxo-4-(piperazin-1-yl)butyl)-3,5,6-trimethylcyclohexa-2,5-diene-1,4-dione;
2-(4-(4-fluoropiperidin-1-yl)-3-hydroxy-3-methyl-4-oxobutyl)-3,5,6-trimethylcyclohexa-2,5-diene-1,4-dione;
2-(4-(4,4-difluoropiperidin-1-yl)-3-hydroxy-3-methyl-4-oxobutyl)-3,5,6-trimethylcyclohexa-2,5-diene-1,4-dione;
2-(4-(4-benzoylpiperazin-1-yl)-3-hydroxy-3-methyl-4-oxobutyl)-3,5,6-trimethylcyclohexa-2,5-diene-1,4-dione;
tert-butyl 4-(2-hydroxy-2-methyl-4-(2,4,5-trimethyl-3,6-dioxocyclohexa-1,4-dienyl)butanoyl)piperazine-1-carboxylate;
N-(2-fluorophenethyl)-2-hydroxy-2-methyl-4-(2,4,5-trimethyl-3,6-dioxocyclohexa-1,4-dienyl)butanamide;
N-(3-fluorophenethyl)-2-hydroxy-2-methyl-4-(2,4,5-trimethyl-3,6-dioxocyclohexa-1,4-dienyl)butanamide;
N-(4-fluorophenethyl)-2-hydroxy-2-methyl-4-(2,4,5-trimethyl-3,6-dioxocyclohexa-1,4-dienyl)butanamide N-(2-chlorophenethyl)-2-hydroxy-2-methyl-4-(2,4,5-trimethyl-3,6-dioxocyclohexa-1,4-dienyl)butanamide;
2-hydroxy-N-(4-methoxyphenyl)-2-methyl-4-(2,4,5-trimethyl-3,6-dioxocyclohexa-1,4-dienyl)butanamide;
N-(4-fluorophenyl)-2-hydroxy-2-methyl-4-(2,4,5-trimethyl-3,6-dioxocyclohexa-1,4-dienyl)butanamide;
N-(4-chlorophenyl)-2-hydroxy-2-methyl-4-(2,4,5-trimethyl-3,6-dioxocyclohexa-1,4-dienyl)butanamide;
N-(2-fluorobenzyl)-2-hydroxy-2-methyl-4-(2,4,5-trimethyl-3,6-dioxocyclohexa-1,4-dienyl)butanamide;
N-(3-fluorobenzyl)-2-hydroxy-2-methyl-4-(2,4,5-trimethyl-3,6-dioxocyclohexa-1,4-dienyl)butanamide;
N-(4-fluorobenzyl)-2-hydroxy-2-methyl-4-(2,4,5-trimethyl-3,6-dioxocyclohexa-1,4-dienyl)butanamide;
N-(2-chlorobenzyl)-2-hydroxy-2-methyl-4-(2,4,5-trimethyl-3,6-dioxocyclohexa-1,4-dienyl)butanamide;
N-(3-chlorobenzyl)-2-hydroxy-2-methyl-4-(2,4,5-trimethyl-3,6-dioxocyclohexa-1,4-dienyl)butanamide;
N-(4-chlorobenzyl)-2-hydroxy-2-methyl-4-(2,4,5-trimethyl-3,6-dioxocyclohexa-1,4-dienyl)butanamide;
2-(3-hydroxy-3-methylbutyl)-5,6-dimethyl-3-phenethylcyclohexa-2,5-diene-1,4-dione;
2-benzyl-3-(3-hydroxy-3-methylbutyl)-5,6-dimethylcyclohexa-2,5-diene-1,4-dione;
2-(3-hydroxy-3-methylbutyl)-5,6-dimethyl-3-(3-phenylpropyl)cyclohexa-2,5-diene-1,4-dione;
2-(3-hydroxy-3-methylbutyl)-5,6-dimethyl-3-(4-(trifluoromethyl)phenyl)cyclohexa-2,5-diene-1,4-dione;
2-(3-hydroxy-3-methylbutyl)-5,6-dimethyl-3-(naphthalen-2-yl)cyclohexa-2,5-diene-1,4-dione;
2-(benzofuran-2-yl)-3-(3-hydroxy-3-methylbutyl)-5,6-dimethylcyclohexa-2,5-diene-1,4-dione;
2-(4-chlorophenyl)-3-(3-hydroxy-3-methylbutyl)-5,6-dimethylcyclohexa-2,5-diene-1,4-dione;
2-(4-ethylphenyl)-3-(3-hydroxy-3-methylbutyl)-5,6-dimethylcyclohexa-2,5-diene-1,4-dione;
2-(3-hydroxy-3-methylbutyl)-5,6-dimethyl-3-(3-(trifluoromethyl)phenyl)cyclohexa-2,5-diene-1,4-dione;
2-(4-tert-butylphenyl)-3-(3-hydroxy-3-methylbutyl)-5,6-dimethylcyclohexa-2,5-diene-1,4-dione;
2-(4-fluorophenyl)-3-(3-hydroxy-3-methylbutyl)-5,6-dimethylcyclohexa-2,5-diene-1,4-dione;
2-(3-fluorophenyl)-3-(3-hydroxy-3-methylbutyl)-5,6-dimethylcyclohexa-2,5-diene-1,4-dione;
2-(3-hydroxy-3-methylbutyl)-3,5-dimethyl-6-(4-(trifluoromethyl)phenyl)cyclohexa-2,5-diene-1,4-dione;
2-(3-hydroxy-3-methylbutyl)-6-(4-methoxyphenyl)-3,5-dimethylcyclohexa-2,5-diene-1,4-dione;
2-(3,4-difluorophenyl)-6-(3-hydroxy-3-methylbutyl)-3,5-dimethylcyclohexa-2,5-diene-1,4-dione;
2-(4-fluorophenyl)-6-(3-hydroxy-3-methylbutyl)-3,5-dimethylcyclohexa-2,5-diene-1,4-dione;
2-(3-hydroxy-3-methylbutyl)-3-(4-methoxyphenyl)-5,6-dimethylcyclohexa-2,5-diene-1,4-dione;
2-(3,5-bis(trifluoromethyl)phenyl)-3-(3-hydroxy-3-methylbutyl)-5,6-dimethylcyclohexa-2,5-diene-1,4-dione;
2-(4-chlorophenyl)-6-(3-hydroxy-3-methylbutyl)-3,5-dimethylcyclohexa-2,5-diene-1,4-dione;
   and all stereoisomers, mixture of stereoisomers, prodrugs, metabolites, salts, phosphate substituted form, crystalline form, non-crystalline form, hydrate or solvate thereof.

Some other redox active compounds encompassed in the invention, are alpha-tocotrienol, beta-tocotrienol and gamma-tocotrienol and all stereoisomers, mixture of stereoisomers, prodrugs, metabolites, salts, phosphate substituted form, crystalline form, non-crystalline form, hydrate or solvate thereof.

### Tests for Diagnosing Hearing Impairment

Tests are known and available for diagnosing hearing impairments. Neuro-otological, neuro-ophthalmological, neurological examinations, and electro-oculography can be used. (Wennmo et al. Acta Otolaryngol (1982) 94:507-15). Sensitive and specific measures are available to identify patients with auditory impairments. For example, tuning fork tests can be used to differentiate a conductive from a sensorineural hearing loss and determine whether the loss is unilateral. An audiometer is used to quantify hearing loss, measured in decibels. With this device the hearing for each ear is measured, typically from 125 to 8000 Hz, and plotted. The speech recognition threshold, the intensity at which speech is recognized as a meaningful symbol, can be determined at various speech frequencies. Speech or phoneme discrimination can also be determined and used as an indicator of sensorineural hearing loss since analysis of speech sounds relies upon the inner ear and the 8^{th} nerve. Tympanometry can be used to diagnose conductive hearing loss and aid in the diagnosis of those patients with sensorineural hearing loss. Electrocochleography, measuring the cochlear microphonic response and action potential of the 8.sup.th nerve, and evoked response audiometry, measured evoked response from the brainstem and auditory cortex, to acoustic stimuli can be used in patients, particularly infants and children or patients with sensorineural hearing loss of obscure etiology. These tests serve a diagnostic function as well as a clinical function in assessing response to therapy.

Sensory and neural hearing losses can be distinguished based on tests for recruitment (an abnormal increase in the perception of loudness or the ability to hear loud sounds normally despite a hearing loss), sensitivity to small increments in intensity, and pathologic adaptation, including neural hearing loss. In sensory hearing loss, the sensation of loudness in the affected ear increases more with each increment in intensity than it does in the normal ear. Sensitivity to small increments in intensity can be demonstrated by presenting a continuous tone of 20 dB above the hearing threshold and increasing the intensity by 1 dB briefly and intermittently. The percentage of small increments detected yields the “short increment sensitivity index” value. High values, 80 to 100%, are characteristic of sensory hearing loss, whereas a neural lesion patient and those with normal hearing cannot detect such small changes in intensity. Pathologic adaptation is demonstrated when a patient cannot continue to perceive a constant tone above threshold of hearing, also known as tone decay. A Bekesy automatic audiometer or equivalent can be used to determine these clinical and diagnostic signs; audiogram patterns of the Type II pattern, Type III pattern and Type IV pattern are indicative of preferred hearing losses suitable for the treatment methods of the invention. As hearing loss can often be accompanied by vestibular impairment, vestibular function can be tested, particularly when presented with a sensorineural hearing loss of unknown etiology.

When possible, diagnostics for hearing loss, such as audiometric tests, should be performed prior to exposure in order to obtain a patient's normal hearing baseline. Upon exposure, particularly to an ototoxic drug, audiometric tests should be performed twice a week and testing should be continued for a period after cessation of the ototoxic drug treatment, since hearing loss may not occur until several days after cessation. U.S. Pat. No. 5,546,956 provides methods for testing hearing that can be used to diagnose the patient and monitor treatment. U.S. Pat. No. 4,637,402 provides a method for quantitatively measuring a hearing defect that can be used to diagnose the patient and monitor treatment.

Another diagnostic test for hearing loss is provided by Athena Diagnostics Inc (Worcester, Mass. 01605). Their OtoDX™ Aminoglycoside Hypersensitivity Test (#327) diagnoses sensorineural, nonsyndromic hearing loss often associated with aminoglycoside antibiotic exposure.

### In Vitro System for Drug Ototoxicity Screening

The conditionally immortalized auditory HEI-OC1 cell line from long-term cultures of transgenic mice Immortomouse™ cochleas has been described in Kalinec, G. et al., Audiol. Nerootol. 2003; 8, 177-189/. It provides a powerful tool for the in vitro study of auditory cells. These cells are more sensitive to aminoglycoside-induced apoptosis that cells from fibroblastic origin. As described in So, H. S., Hearing Research (2005) 204, 127-139 and in Devarajan et al., Hearing Research (2002), 174 45-54, HEI-OC1 cells are maintained in high glucose Dulbecco's modified Eagle medium (DMEM) containing 10% FBS under permissive conditions, 33° C., 10% CO₂. Cells are incubated with varying concentrations of platinum-containing antineoplastic agents, such as cisplatin and its analogs or aminoglycoside antibiotics such as gentamicin and its analogs, for different time periods. Cells incubated in diluent alone were the controls.

### In Vivo Systems for Drug Ototoxicity Screening

There is a wide range of animal models, which can be used to explore the nature of deafness. Rodents provide models for NIHL, drug induced hearing loss, specific loss of SGNs, progressive and age-related hearing loss.

Auditory brainstem response (ABR) is a screening test that can be given to both humans and animals to monitor for hearing loss or deafness. It is a method employed to assess the functions of the ears, cranial nerves, and various brain functions of the lower part of the auditory system. It is a safe and painless test of auditory pathway and brainstem function in response to auditory or (click) stimuli. Noise induced ABR threshold shifts are assessed at each test frequency following noise exposure.

Missing hair cells are another screen to evaluate the loss of hearing. Missing hair cells of sacrificed rodents are counted in rhodamine phalloidin-labeled surface preparations and the percentage of inner hair cell and outer hair cell loss can quantitatively be evaluated during treatment.

### Pharmaceutical Formulations

The compounds described herein can be formulated as pharmaceutical compositions by formulation with additives such as pharmaceutically acceptable excipients, pharmaceutically acceptable carriers, and pharmaceutically acceptable vehicles. Suitable pharmaceutically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003) and 21st edition (2005), incorporated herein by reference.

A pharmaceutical composition can comprise a unit dose formulation, where the unit dose is a dose sufficient to have a therapeutic or suppressive effect or an amount effective to modulate, normalize, or enhance an energy biomarker. The unit dose may be sufficient as a single dose to have a therapeutic or suppressive effect or an amount effective to modulate, normalize, or enhance an energy biomarker. Alternatively, the unit dose may be a dose administered periodically in a course of treatment or suppression of a disorder, or to modulate, normalize, or enhance an energy biomarker.

Pharmaceutical compositions containing the compounds of the invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

Time-release or controlled release delivery systems may be used, such as a diffusion controlled matrix system or an erodible system, as described for example in: Lee, “Diffusion-Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York 1992. The matrix may be, for example, a biodegradable material that can degrade spontaneously in situ and in vivo for, example, by hydrolysis or enzymatic cleavage, e.g., by proteases. The delivery system may be, for example, a naturally occurring or synthetic polymer or copolymer, for example in the form of a hydrogel. Exemplary polymers with cleavable linkages include polyesters, polyorthoesters, polyanhydrides, polysaccharides, poly(phosphoesters), polyamides, polyurethanes, poly(imidocarbonates) and poly(phosphazenes).

The compound of the present invention can also be delivered by implanting a sustained-release drug delivery device in the inner ear as described Ashton, P. et al in US Pat. Publication No. 2007/0160648.

The compound of the present invention can also be delivered with a device which is a wick-like ontological implant for delivery of medicament to a treatment site in the inner ear as described by Silverstein, H. in U.S. Pat. No. 6,120,484.

Another type of device used for sustained release of a drug to the ear, described by Zenner et al. in U.S. Pat. No. 5,895,372 is an implantable dosaging system for administration in a form of dissolved or suspended fluids using a pump mechanism.

Another treatment system described in U.S. Pat. No. 5,474,529 is an apparatus for use in the middle and inner ear using a diffusion mechanism.

The compounds of the invention may be administered enterally, orally, parenterally, sublingually, by inhalation (e.g. as mists or sprays), rectally, or topically in dosage unit formulations containing conventional nontoxic pharmaceutically acceptable carriers, adjuvants, and vehicles as desired. For example, suitable modes of administration include oral, subcutaneous, transdermal, transmucosal, iontophoretic, intravenous, intraarterial, intramuscular, intraperitoneal, intranasal (e.g. via nasal mucosa), subdural, rectal, gastrointestinal, and the like, and directly to a specific or affected organ or tissue. For delivery to the central nervous system, spinal and epidural administration, or administration to cerebral ventricles, can be used. Topical administration may also involve the use of transdermal administration such as transdermal patches or iontophoresis devices. The term parenteral as used herein includes subcutaneous injections, intravenous, intramuscular, intrastemal injection, or infusion techniques. The compounds are mixed with pharmaceutically acceptable carriers, adjuvants, and vehicles appropriate for the desired route of administration. Oral administration is a preferred route of administration, and formulations suitable for oral administration are preferred formulations. The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The compounds can also be administered in liposome formulations. The compounds can also be administered as prodrugs, where the prodrug undergoes transformation in the treated subject to a form which is therapeutically effective. Additional methods of administration are known in the art.

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq (1976).

The invention also provides articles of manufacture and kits containing materials useful for treating or suppressing hearing impairment. The invention also provides kits comprising any one or more of the redox-active compounds. In some embodiments, the kit of the invention comprises the container described above.

The invention also provides articles of manufacture and kits containing materials useful for treating or suppressing hearing or balance impairment. The invention also provides kits comprising any one or more of the redox-active compounds in combination with an aminoglycoside such as gentamicin. In some embodiments, the kit of the invention comprises the container described above.

In other aspects, the kits may be used for any of the methods described herein, including, for example, to treat an individual with a hearing impairment, or to suppress a hearing impairment in an individual.

The amount of active ingredient that may be combined with the carrier materials to produce a single dosage form will vary depending upon the host to which the active ingredient is administered and the particular mode of administration. It will be understood, however, that the specific dose level for any particular patient will depend upon a variety of factors including the activity of the specific compound employed, the age, body weight, body area, body mass index (BMI), general health, sex, diet, time of administration, route of administration, rate of excretion, drug combination, and the type, progression, and severity of the particular disease undergoing therapy. The pharmaceutical unit dosage chosen is usually fabricated and administered to provide a defined final concentration of drug in the blood, tissues, organs, or other targeted region of the body. The therapeutically effective amount or effective amount for a given situation can be readily determined by routine experimentation and is within the skill and judgment of the ordinary clinician.

Examples of dosages which can be used are an effective amount within the dosage range of about 0.1 mg/kg to about 300 mg/kg body weight, or within about 1.0 mg/kg to about 100 mg/kg body weight, or within about 1.0 mg/kg to about 50 mg/kg body weight, or within about 1.0 mg/kg to about 30 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight. Compounds of the present invention may be administered in a single daily dose, or the total daily dosage may be administered in divided dosage of two, three or four times daily

While the compounds of the invention can be administered as the sole active pharmaceutical agent, they can also be used in combination with one or more other agents with ototoxic side effects. When additional active agents are used in combination with the compounds of the present invention, the additional active agents may generally be employed in therapeutic amounts as indicated in the Physicians' Desk Reference (PDR) 53rd Edition (1999), which is incorporated herein by reference, or such therapeutically useful amounts as would be known to one of ordinary skill in the art.

The compounds of the invention and the other therapeutically active agents can be administered at the recommended maximum clinical dosage or at lower doses. Dosage levels of the active compounds in the compositions of the invention may be varied so as to obtain a desired therapeutic response depending on the route of administration, severity of the disease and the response of the patient. When administered in combination with other therapeutic agents, the therapeutic agents can be formulated as separate compositions that are given at the same time or different times, or the therapeutic agents can be given as a single composition.

### BIOLOGICAL EXAMPLE

### In Vitro Ototoxicity Screening

The conditionally immortalized auditory HEI-OC1 cells from long-term cultures of transgenic mice Immortomouse™ cochleas as described in Kalinec, G. et al., Audiol. Nerootol. 2003; 8, 177-189/. were maintained in high glucose Dulbecco's modified Eagle medium (DMEM) containing 10% FBS under permissive conditions, 33° C., 10% CO₂. Cells were pretreated overnight with compounds, and apoptosis was detected by caspase3/7 activity after 24 hours of 50 uM cisplatin incubation. Cells incubated in diluent alone were the controls. Compounds of the present invention exhibited an EC₅₀ of less than about 100 nM.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A method for preventing or treating a mammal having or prone to having a hearing or a balance impairment, said method comprising administering to the mammal a therapeutically effective amount of a redox-active therapeutic, with the proviso that the redox-active therapeutic is not Idebenone, Vitamin E, or Trolox.

2. The method of claim 1, wherein the redox-active therapeutic comprises a compound selected from Formula I, Formula II, Formula III, Formula IV, Formula V, and Formula VI.

3. The method of claim 1, wherein the redox-active therapeutic comprises a compound selected from Formula I, Formula II, Formula III, and Formula IV with the following structures: 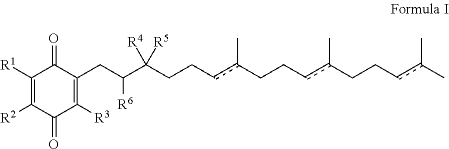
wherein,
the bonds indicated with a dashed line can independently be single or double,
R¹, R², and R³ are independently selected from H, (C₁-C₄)-alkyl, (C₁-C₄)-haloalkyl, CN, F, Cl, Br, and I; and
R⁴ is independently selected from hydroxy and (C₁-C₄)-alkyl, R⁵ is independently selected from (C₁-C₄)-alkyl, and R⁶ is hydrogen; or
R⁴ is alkyl, and R⁵ and R⁶ are hydrogen; or
R⁴ is alkyl, and R⁵ and R⁶ together form a double bond;
or a stereoisomer, mixture of stereoisomers, a salt, a hydrate, or a solvate thereof; 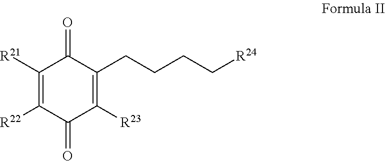
wherein,
R²¹, R²², and R²³ are independently selected from H, (C₁-C₄)-alkyl, (C₁-C₄)-haloalkyl, CN, F, Cl, Br, and I;
R²⁴ is independently selected from (C₁-C₂₀)-alkyl, (C₁-C₂₀)-alkenyl, (C₁-C₂₀)-alkynyl, and (C₁-C₂₀) containing at least one double bond and at least one triple bond,
or a stereoisomer, mixture of stereoisomers, a salt, a hydrate, or a solvate thereof; 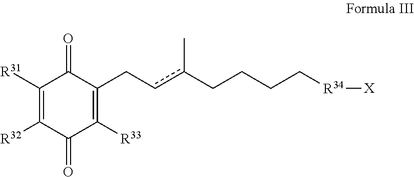
wherein,
the bond indicated with a dashed line can be single or double;
R³¹, R³², and R³³ are independently selected from the group consisting of H, (C₁-C₅)-alkyl, (C₁-C₅)-haloalkyl, (C₂-C₅)-alkenyl, (C₂-C₅)-haloalkenyl, (C₂-C₅)-alkynyl, —(C₁-C₅)-haloalkynyl, OR³⁵, SR³⁵, CN, F, Cl, Br, I, N₃, and NR³⁵R³⁶; where R³⁵ and R³⁶ are independently selected from the group consisting of H, (C₁-C₅)-alkyl, (C₃-C₅)-cycloalkyl, (C₁-C₅)-haloalkyl, aryl, heteroaryl, —(C═O)—(C₁-C₈)-alkyl, and —(C═O—(C₀-C₈)-alkyl-(C₆-C₁₀)-aryl-(C₀-C₈)-alkyl, or where R³⁵ and R³⁶ selected from these groups are combined to form a ring;
R³⁴ represents a linear or branched group containing 1 to 32 carbon atoms and any number of single, double, or triple bonds in any chemically possible combination;
X is selected from the group consisting of H, F, Cl, Br, I, CN, —N₃, —NR³⁷R³⁸, and —OR³⁹; where R³⁷ and R³⁸ are independently selected from H, (C₁-C₈)-alkyl or (C₁-C₈)-haloalkyl, —(C═O)—(C₁-C₈)-alkyl, or where either one of R³⁷ and R³⁸ are independently selected from the group consisting of —(C═O)—(C₁-C₈)-haloalkyl; —(C═O)—NH₂; —(C═O)—NH(C₁-C₈)-alkyl; —(C═O)—NH(C₁-C₈)-haloalkyl; —(C═O)—NR³⁰¹R³⁰², where R³⁰¹ and R³⁰² together with the nitrogen atom to which they are attached combine to form a 3- to 8-membered ring, and where another group selected from —NH—, —N((C₁-C₄)-alkyl)-, —O—, or —S— can be optionally incorporated in the ring formed by R³⁰¹ and R³⁰² and the nitrogen atom to which they are attached; —(C═O)—O—(C₁-C₈)-alkyl, —(C═O)—O—(C₁-C₈)-haloalkyl, —S(O)₂—(C₁-C₈)-alkyl, —S(O)₂-aryl, and —S(O)₂-aralkyl, and where the other of R³⁷ or R³⁸ is H, (C₁-C₈)-alkyl or (C₁-C₈)-haloalkyl or where R³⁷ and R³⁸ selected from these groups together with the nitrogen atom to which they are attached combine to form a 3- to 8-membered ring, and where another group selected from —NH—, —N((C₁-C₄)-alkyl)-, —O—, or —S— can be optionally incorporated in the ring formed by R³⁷ and R³⁸ and the nitrogen atom to which they are attached; where R³⁹ is independently selected from H, —(C₁-C₈)-alkyl or (C₁-C₈)-haloalkyl, —(C═O)—(C₁-C₈)-alkyl, —(C═O)-(C₁-C₈)-haloalkyl, —(C═O)—NH₂, —(C═O)—NH—(C₁-C₈)-alkyl, —(C═O)—NH(C₁-C₈)-haloalkyl, —(C═O)—NR³⁰¹R³⁰² where R³⁰¹ and R³⁰² together with the nitrogen atom to which they are attached combine to form a 3- to 8-membered ring, and where another group selected from —NH—, —N((C₁-C₄)-alkyl)-, —O—, or —S— can be optionally incorporated in the ring formed by R³⁰¹ and R³⁰² and the nitrogen atom to which they are attached, —(C═O)—O—(C₁-C₈)-alkyl, —(C═O)—O—(C₁-C₈)-haloalkyl, —S(O)₂—(C₁-C₅)-alkyl, —S(O)₂-aryl, and —S(O)₂-aralkyl; with the proviso that when both of R³¹ and R³² are —OCH₃ and R³³ is —CH₃, then X is not —H or —OH;
or a stereoisomer, mixture of stereoisomers, a salt, a hydrate, or a solvate thereof; 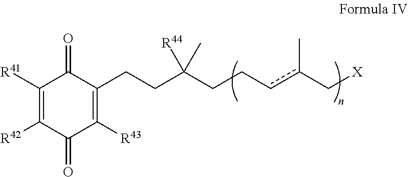
wherein,
n is an integer from 0 to 9 inclusive, and each unit can be the same or different;
the bond(s) indicated by a dashed line can independently of each other be single or double bonds;
R⁴¹, R⁴², and R⁴³ are independently selected from the group consisting of H, (C₁-C₅)-alkyl, (C₁-C₅)-haloalkyl, (C₂-C₅)-alkenyl, (C₂-C₅)-haloalkenyl, (C₂-C₅)-alkynyl, (C₂-C₅)-haloalkynyl, —OR⁴⁵, —SR⁴⁵, CN, F, Cl, Br, I, N₃, and —NR⁴⁵R⁴⁶; where R⁴⁵ and R⁴⁶ are independently selected from the group consisting of H, (C₁-C₅)-alkyl, (C₃-C₆)-cycloalkyl, (C₁-C₅)-haloalkyl, aryl, heteroaryl, —(C═O)—(C₁-C₈)-alkyl, and —(C═O)—(C₀-C₈)-alkyl-(C₆-C₁₀)aryl-(C₀-C₄)alkyl, or where R⁴⁵ and R⁴⁶ selected from these groups are combined to form a ring;
R⁴⁴ is selected from the group consisting of H, —OR⁴⁵, —SR⁴⁵, F, Cl, Br, I, and —NR⁴⁵R⁴⁶;
X is selected from the group consisting of H, —NR⁴⁷R⁴⁸, —OR⁴⁹ and —(CH₂)₂C(CH₃)₂OH;
R⁴⁷ and R⁴⁸ are independently selected from H, —(C₁-C₈)-alkyl or (C₁-C₈)haloalkyl, —(C═O)—(C₁-C₈)-alkyl, or where either one of R⁴⁷ and R⁴⁸ are independently selected from the group consisting of —(C═O)—(C₁-C₈)-haloalkyl, —(C═O)—NH₂, —(C═O)—(C₁-C₈)alkyl, —(C═O)—NH(C₁-C₈)-haloalkyl, —(C═O)—NR⁴⁰¹R⁴⁰² where R⁴⁰¹ and R⁴⁰² together with the nitrogen atom to which they are attached combine to form a 3- to 8-membered ring, and where another group selected from —NH—, —N((C₁-C₄)alkyl)-, —O—, or —S— can be optionally incorporated in the ring formed by R⁴⁰¹ and R⁴⁰² and the nitrogen atom to which they are attached; —(C═O)—O—(C₁-C₈)alkyl, —(C═O)—O(C₁-C₅)-haloalkyl, —S(O)₂—(C₀-C₈)-alkyl, —S(O)₂-aryl, and —S(O)₂-aralkyl, and where the other of R⁴⁷ or R⁴⁸ is H, (C₁-C₈)-alkyl or (C₁-C₈)-haloalkyl or where R⁴⁷ and R⁴⁸ selected from these groups are combined to form a ring, or where R⁴⁷ and R⁴⁸ together with the nitrogen atom to which they are attached combine to form a 3- to 8-membered ring, and where another group selected from —NH—, —N((C₁-C₄)-alkyl)-, —O—, or —S— can be optionally incorporated in the ring formed by R⁴⁷ and R⁴⁸ and the nitrogen atom to which they are attached; where R⁴⁹ is independently selected from H, (C₁-C₈)-alkyl or (C₁-C₈)-haloalkyl, —(C═O)—(C₁-C₈)-alkyl, —(C═O)—(C₁-C₈)haloalkyl, —(C═O)—NH₂, —(C═O)—(C₁-C₅)-alkyl, —(C═O)—NH(C₁-C₈)-haloalkyl, —(C═O)—NR⁴⁰¹R⁴⁰² where R⁴⁰¹ and R⁴⁰² together with the nitrogen atom to which they are attached combine to form a 3- to 8-membered ring, and where another group selected from —NH—, —N((C₁-C₄)-alkyl)-, —O—, or —S— can be optionally incorporated in the ring formed by R⁴⁰¹ and R⁴⁰² and the nitrogen atom to which they are attached;—(C═O)—(C₁-C₈)-alkyl, —(C═O)—O(C₁-C₈)-haloalkyl, —S(O)₂(C₁-C₈)-alkyl, —S(O)₂-aryl, and —S(O)₂-aralkyl; with the provisos that when n=3 and if R⁴⁴ is —H or —OH, then X is not —H, and that when R⁴¹ and R⁴² are —OCH₃ and R⁴³ is —CH₃, then either R⁴⁴ is neither H nor —OH, or X is neither H nor —OH nor —(CH2)₂C(CH₃)₂OH;
or a stereoisomer, mixture of stereoisomers, a salt, a hydrate, or a solvate thereof.

4. The method of claim 1, wherein the redox-active therapeutic comprises a compound selected from alpha tocopherol quinone, beta tocopherol quinone, gamma tocopherol quinone, alpha tocotrienol quinone, beta tocotrienol quinone, and gamma tocotrienol quinone, or mixtures thereof.

5. The method of claim 1, wherein the redox-active therapeutic comprises a compound of Formula V: 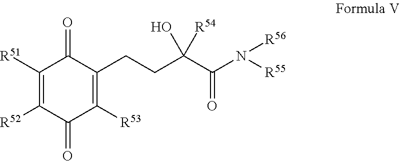
wherein,
R⁵¹, R⁵², and R⁵³ are independently selected from hydrogen and (C₁-C₆)-alkyl;
R⁵⁴ is (C₁-C₆)-alkyl;
R⁵⁵ and R⁵⁶ are independently selected from hydrogen, hydroxy, alkoxy, (C₁-C₄₀)-alkyl, (C₁-C₄₀)-alkenyl, (C₁-C₄₀)-alkynyl, and aryl, with the proviso that only one of R⁵⁵ and R⁵⁶ is hydroxy; where the alkyl, alkenyl, alkynyl or aryl groups may optionally be substituted with
OR⁵⁰¹, —S(O)₀₋₂R⁵⁰¹, —CN, —F, —Cl, —Br, —I, —NR⁵⁰¹R⁵⁰², oxo, (C₃-C₆)-cycloalkyl, aryl, aryl-(C₁-C₆)-alkyl, heteroaryl, heterocyclyl, —C(═O)—R⁵⁰³, —C(═O)—(C₀-C₆)-alkyl-aryl, —C(═O)—O—R⁵⁰³, —C(═O)—O—(C₀-C₆)-alkyl-aryl, —C(═O)—N—R⁵⁰³R⁵⁰⁴, C(═O)—N—(C₀-C₆)-alkyl-aryl, —N—C(═O)—R⁵⁰³, —N—C(═O)—(C₀-C₆)-alkyl-aryl; where the aryl, heteroaryl and heterocyclyl ring substituents may be further substituted with (C₁-C₆)-alkyl, (C₁-C₆)-haloalkyl, oxo, hydroxy, (C₁-C₆)-alkoxy, —C(═O)—(C₁-C₆)-alkyl and —C(═O)—O—(C₁-C₆)-alkyl; and where one of the carbons of the alkyl, alkenyl, or alkynyl groups may be replaced by a heteroatom selected from O, N or S; or
R⁵⁵ and R⁵⁶ together with the atom to which they are attached form a saturated or unsaturated 3-8 membered ring, optionally incorporating one or more additional heteroatoms independently selected from one, two, or three N, O, or S atoms, optionally substituted with oxo, —OR⁵⁰¹, —SR⁵⁰¹, —CN, —F, —Cl, —Br, —I, —NR⁵⁰¹R⁵⁰², (C₁-C6)-alkyl, (C₁-C₆)-haloalkyl; hydroxy-(C₁-C₆)-alkyl, —C(═O)—H, —C(═O)—(C₁-C₆)-alkyl, —C(═O)-aryl, —C(═O)—OH, or —C(═O)—O—(C₁-C₆)-alkyl; or
R⁵⁵ and R⁵⁶ together with the nitrogen atom to which they are attached form a N,N′-disubstituted piperazine where the nitrogen substitution at the 4-position is a group identical to the substitution at the 1-position forming a compound of formula Va, where R⁵¹, R⁵², R⁵³, and R⁵⁴ are as defined above: 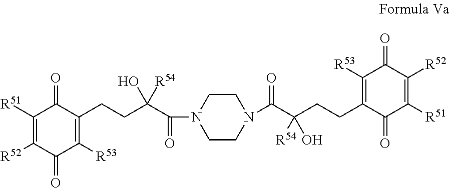
R⁵⁰¹ and R⁵⁰² are independently selected from the group consisting of hydrogen, (C₁-C₆)-alkyl, (C₁-C₆)-haloalkyl, aryl, aryl-(C₁-C ₆)-alkyl, hetero aryl, heterocyclyl, —C(═O)—H, —C(═O)—(C₁-C₆)-alkyl, —C(═O)-aryl and —C(═O)-(C₁-C₆)-alkyl-aryl; and
R⁵⁰³ and R⁵⁰⁴ are selected from hydrogen and (C₁-C₆)-alkyl;
or a stereoisomer, mixture of stereoisomers, a salt, a hydrate, or a solvate thereof.

6. The method of claim 5, wherein the redox-active therapeutic comprises a compound selected from 2-(3-hydroxy-4-(4-hydroxypiperidin-1-yl)-3-methyl-4-oxobutyl)-3,5,6-trimethylcyclohexa-2,5-diene-1,4-dione, 2-hydroxy-2-methyl-4-(2,4,5-trimethyl-3,6-dioxocyclohexa-1,4-dienyl)butanamide, 2-(4-(4-acetylpiperazin-1-yl)-3-hydroxy-3-methyl-4-oxobutyl)-3,5,6-trimethylcyclohexa-2,5-diene-1,4-dione, N-(2-(dimethylamino)ethyl)-2-hydroxy-2-methyl-4-(2,4,5-trimethyl-3,6-dioxocyclohexa-1,4-dienyl)butanamide, and 2-(3-hydroxy-3-methyl-4-(4-methylpiperazin-1-yl)-4-oxobutyl)-3,5,6-trimethylcyclohexa-2,5-diene-1,4-dione.

7. The method of claim 1, wherein the redox-active therapeutic comprises a compound of Formula VI: 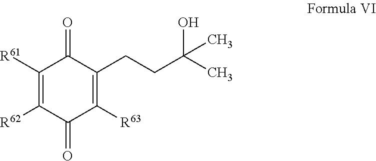
wherein,
R⁶¹ is aryl-(C₀-C₆)-alkyl- or heterocyclyl-(C₀-C₆)-alkyl-, wherein the aryl or heterocyclyl is optionally substituted with one or more substituents selected from (C₁-C₆)-alkyl, (C₂-C₆)-alkenyl, (C₂-C₆)-alkynyl, halogen, (C₁-C₆)-haloalkyl, hydroxy, (C₁-C₆)-alkoxy, CN, nitro, —C(═O)OR⁶⁴, —NR⁶⁵R⁶⁶, —C(═O)NR⁶⁵R⁶⁶, —SH, (C₁-C₆)-thioalkyl, and —C(═O)R⁶⁴; and wherein the (C₀-C₆)-alkyl group is optionally substituted with OH, —O—(C₁-C₄)-alkyl, —NH₂, —NH(C₁-C₄)-alkyl, —N ((C₁-C₄)-alkyl)₂, oxo or halogen; and
R⁶² and R⁶³ are independently selected from hydrogen, halogen, (C₁-C₆)-alkyl and (C₁-C₆)-alkoxy; or
R⁶³ is aryl-(C₀-C₆)-alkyl- or heterocyclyl-(C₀-C₆)-alkyl-, wherein the aryl or heterocyclyl is optionally substituted with one or more substituents selected from (C₁-C₆)-alkyl, (C₂-C₆)-alkenyl, (C₂-C₆)-alkynyl, halogen, (C₁-C₆)-haloalkyl-, hydroxy, (C₁-C₆)-alkoxy, CN, nitro, —C(═O)OR⁶⁴, —NR⁶⁵R⁶⁶, —C(═O)NR⁶⁵R⁶⁶, —SH, (C₁-C₆)-thioalkyl-, and C(═O)R⁶⁴; and wherein the (C₀-C₆)-alkyl group is optionally substituted with OH, —O(C₁-C₄)-alkyl, —NH₂, —NH(C₁-C₄)-alkyl, —N ((C₁-C₄)-alkyl)₂, oxo or halogen; and
R⁶¹ and R⁶² are independently selected from hydrogen, halogen, (C₁-C₆)-alkyl, and (C₁-C₆)-alkoxy;
R⁶⁴ is hydrogen, (C₁-C₆)-alkyl, aryl, or aryl-(C₁-C₆)-alkyl-;
R⁶⁵ and R⁶⁶ are independently of each other hydroxy, (C₁-C₆)-alkoxy, (C₁-C₆)-alkyl, (C₂-C₆)-alkenyl, (C₂-C₆)-alkynyl, aryl, aryl-(C₁-C₆)-alkyl-, heterocyclyl, or heterocyclyl-(C₁-C₆)-alkyl-; wherein the alkyl, alkenyl, alkynyl, aryl and heterocyclyl groups can be further substituted with oxo, halogen, (C₁-C₆)-haloalkyl, hydroxy, (C₁-C₆)-alkoxy, or —C(═O)OR⁶⁴;
or a stereoisomer, mixture of stereoisomers, a salt, a hydrate, or a solvate thereof.

8. The method of claim 7, wherein the redox-active therapeutic comprises a compound selected from 2-(3-hydroxy-3-methylbutyl)-5,6-dimethyl-3-(4-(trifluoromethyl)phenyl)cyclohexa-2,5-diene-1,4-dione, 2-(3-hydroxy-3-methylbutyl)-5,6-dimethyl-3-(4-(trifluoromethyl)phenyl)cyclohexa-2,5-diene-1,4-dione, 2-(3-hydroxy-3-methylbutyl)-3,5-dimethyl-6-(4-(trifluoromethyl)phenyl)cyclohexa-2,5-diene-1,4-dione, and 2-(4-chlorophenyl)-6-(3-hydroxy-3-methylbutyl)-3,5-dimethylcyclohexa-2,5-diene-1,4-dione.

9. The method of claim 1, wherein the redox-active therapeutic consists essentially of alpha tocotrienol, beta tocotrienol, gamma tocotrienol or mixtures thereof.

10. The method of claim 1, wherein the impairment is a result of aging.

11. The method of claim 1, wherein the impairment is a result of neuronal damage.

12. The method of claim 1, wherein the impairment is a result of noise or of acoustic trauma.

13. The method of claim 12, where the impairment is tinnitus.

14. The method of claim 11, wherein said damage is caused by an ototoxic agent.

15. The method of claim 14, wherein said ototoxic agent is a pharmaceutical drug selected from the group consisting of an aminoglycoside antibiotic, a chemotherapeutic agent, a salicylate or salicylate-like compound, a diuretic and a quinine.

16. The method of claim 14, wherein the ototoxic agent is an anti-neoplastic agent selected from cisplatin and a cisplatin-like compound.

17. The method of claim 16, wherein the redox-active therapeutic comprises a compound selected from Formula I, Formula II, Formula III, Formula IV, Formula V, and Formula VI.

18. The method of claim 16, wherein the redox-active therapeutic comprises a compound selected from Formula I, Formula II, Formula III, and Formula IV, as described in claim 3.

19. The method of claim 18, wherein the redox-active therapeutic comprises a compound selected from alpha tocopherol quinone, beta tocopherol quinone, gamma tocopherol quinone, alpha tocotrienol quinone, beta tocotrienol quinone, and gamma tocotrienol quinone, or mixtures thereof.

20. The method of claim 16, wherein the redox-active therapeutic comprises a compound of Formula V, as described in claim 5.

21. The method of claim 20, wherein the redox-active therapeutic is a compound selected from 2-(3-hydroxy-4-(4-hydroxypiperidin-1-yl)-3-methyl-4-oxobutyl)-3,5,6-trimethylcyclohexa-2,5-diene-1,4-dione, 2-hydroxy-2-methyl-4-(2,4,5-trimethyl-3,6-dioxocyclohexa-1,4-dienyl)butanamide, 2-(4-(4-acetylpiperazin-1-yl)-3-hydroxy-3-methyl-4-oxobutyl)-3,5,6-trimethylcyclohexa-2,5-diene-1,4-dione, N-(2-(dimethylamino)ethyl)-2-hydroxy-2-methyl-4-(2,4,5-trimethyl-3,6-dioxocyclohexa-1,4-dienyl)butanamide, and 2-(3-hydroxy-3-methyl-4-(4-methylpiperazin-1-yl)-4-oxobutyl)-3,5,6-trimethylcyclohexa-2,5-diene-1,4-dione.

22. The method of claim 16, wherein the redox-active therapeutic comprises a compound of Formula VI, as described in claim 7.

23. The method of claim 22, wherein the redox-active therapeutic is a compound selected from 2-(3-hydroxy-3-methylbutyl)-5,6-dimethyl-3-(4-(trifluoromethyl)phenyl)cyclohexa-2,5-diene-1,4-dione, 2-(3-hydroxy-3-methylbutyl)-5,6-dimethyl-3-(4-(trifluoromethyl)phenyl)cyclohexa-2,5-diene-1,4-dione, 2-(3-hydroxy-3-methylbutyl)-3,5-dimethyl-6-(4-(trifluoromethyl)phenyl)cyclohexa-2,5-diene-1,4-dione, and 2-(4-chlorophenyl)-6-(3-hydroxy-3-methylbutyl)-3,5-dimethylcyclohexa-2,5-diene-1,4-dione.

24. The method of claim 16, wherein the redox-active therapeutic consists essentially of alpha tocotrienol, beta tocotrienol, gamma tocotrienol or mixtures thereof.

25. The method of claims 1-24, wherein the impairment is a hearing impairment.

26. The method of claims 1-24, wherein the impairment is a balance impairment.

27. A method of reversing hearing loss, or recovering or enhancing hearing function said method comprising administering to the mammal a therapeutically effective amount of a redox-active therapeutic.

28. The method of claim 27, wherein the redox-active therapeutic has a chemical structure comprising a quinone moiety.

29. A therapeutic composition for treating or preventing a hearing or a balance impairment caused by an ototoxic agent in a mammal in need of such treatment, comprising a therapeutic amount of a combination of the ototoxic agent and a redox-active therapeutic.

30. The composition of claim 29, wherein the redox active therapeutic is a compound selected from Formula I, Formula II, Formula III, Formula IV, Formula V, and Formula VI.

31. The therapeutic composition of claim 29, wherein the redox-active therapeutic is selected from alpha tocotrienol, beta tocotrienol, gamma tocotrienol, alpha tocotrienol quinone, beta tocotrienol quinone, and gamma tocotrienol quinone, or mixtures thereof.

32. The therapeutic composition of claim 29, wherein the redox-active therapeutic is selected from essentially pure alpha tocotrienol, essentially pure beta tocotrienol, essentially pure gamma tocotrienol, essentially pure alpha tocotrienol quinone, essentially pure beta tocotrienol quinone, essentially pure gamma tocotrienol quinone, and mixtures thereof.

33. The therapeutic composition of claims 29-32, wherein the ototoxic drug is an anti-neoplastic drug selected from cisplatin and a cisplatin-like compound.
